(19) United States
(12) Patent Application Publication
(10) Pub. No.: US 2023/0404896 A1
(43) Pub. Date: Dec. 21, 2023

(54) Styling Shampoo Compositions

(71) Applicant: BASF SE, Ludwigshafen (DE)

(72) Inventors: Zhihui Zhu, Shanghai (CN); Lan Liao, Shanghai (CN); Yu Ting Xu, Shanghai (CN); Yi Yao Wang, Shanghai (CN); Jun Tao Xia, Shanghai (CN)

(21) Appl. No.: 18/033,859

(22) PCT Filed: Oct. 13, 2021
(86) PCT No.: PCT/EP2021/078351

§ 371 Date: Apr. 26, 2023

(30) Foreign Application Priority Data
Nov. 3, 2020 (WO) PCT/CN2020/126158

(51) Int. Cl. A61K 8/81; A61Q 5/02; A61Q 5/06; A61K 8/44; A61K 8/46

(52) U.S. Cl. CPC A61K 8/8182; A61Q 5/02; A61K 8/463; A61K 8/442; A61Q 5/06

(57) ABSTRACT

Styling shampoo compositions which comprise (a) a cationic polymer with charge density of from 2 to 10 meq/gram; (b) at least one anionic surfactant selected from sulfate anionic surfactants and amino acid anionic surfactants; and (c) optionally an amphoteric or non-ionic surfactant or combination thereof, wherein the amount of (a) is from 0.3 to 10 wt %, based on the weight of the styling shampoo compositions.

### FIELD OF THE INVENTION

The present invention relates to styling shampoo compositions which provide improved styling performance. More particularly, the present invention relates to styling shampoo compositions which comprise a cationic polymer and at least one anionic surfactant to improve the styling performance of the compositions, especially when used in surfactant matrices such as an amphoteric or non-ionic surfactant or combination thereof.

### BACKGROUND OF THE INVENTION

Hair shampoo often provides acceptable cleaning but little or no styling effect. To meet the styling needs, another styling product, especially leave-on styling product like hair wax, styling gel or styling spray is often used.

Recently, styling shampoo compositions have been developed which can provide cleaning and styling performance from a single product.

WO 98/50007 discloses styling shampoo compositions which comprise a surfactant component selected from the group consisting of a combination of an anionic surfactant and an amphoteric surfactant, and a combination of an anionic surfactant and a zwitterionic surfactant; a cationic deposition polymer having a cationic charge density of from about 0.2 meq/gram to about 2 meq/gram and which is selected from the group consisting of cationic cellulose polymers, cationic guar gum derivatives, and mixtures thereof; an organic cationic hair styling polymer having a cationic charge density of greater than about 2 meq/gram to less than about 4.75 meq/gram; and water. In WO 98/50007, cationic deposition polymer and cationic styling polymer are used together to achieve the styling effect which is neither cost and process effective, nor flexible enough from formulation's perspective.

Therefore, there is still a desire for alternative styling shampoo compositions which provide improved styling performance on the basis of the fewest possible feed materials.

### SUMMARY OF THE INVENTION

The present invention relates to styling shampoo compositions which comprise (a) a cationic polymer with charge density of from 2 to 10 meq/gram; (b) at least one anionic surfactant selected from sulfate anionic surfactants and amino acid anionic surfactants; and (c) optionally an amphoteric or non-ionic surfactant or combination thereof, wherein the amount of (a) is from 0.3 to 10 wt %, based on the weight of the styling shampoo compositions.

The present invention also relates to a process for preparing the styling shampoo compositions of the present invention.

The present invention also relates to the use of the styling shampoo compositions of the present invention.

The present invention includes but not limited to the following embodiments:

1. Styling shampoo compositions which comprise (a) a cationic polymer with charge density of from 2 to 10 meq/gram, preferably from 4 to 8 meq/gram, more preferably from 5 to 7 meq/gram; (b) at least one anionic surfactant selected from sulfate anionic surfactants and amino acid anionic surfactants; and (c) optionally an amphoteric or non-ionic surfactant or combination thereof, wherein the amount of (a) is from 0.3 to 10 wt %, preferably from 0.5 to 9 wt %, more preferably from 1 to 8 wt %, even more preferably from 1.5 to 8 wt %, based on the weight of the styling shampoo compositions.

2. The styling shampoo compositions according to embodiment 1, wherein the styling shampoo compositions comprise (a) a cationic polymer with charge density of from 5 to 7; and (b) at least one anionic surfactant selected from sulfate anionic surfactants, wherein the amount of (a) is from 0.3 to 2.5 wt %, preferably from 0.5 to 2.2 wt %, more preferably from 1 to 2 wt %, based on the weight of the styling shampoo compositions.

3. The styling shampoo compositions according to embodiment 1, wherein the styling shampoo compositions comprise (a) a cationic polymer with charge density of from 2 to 10 meq/gram, preferably from 4 to 8 meq/gram, more preferably from 5 to 7 meq/gram; and (b) at least one anionic surfactant selected from amino acid anionic surfactants, wherein the amount of (b) is from 7 to 30 wt %, preferably from 8 to 25 wt %, more preferably from 9 to 15 wt %, based on the weight of the styling shampoo compositions.

4. The styling shampoo compositions according to embodiment 1, wherein the styling shampoo compositions comprise (a) a cationic polymer with charge density of from 2 to 10 meq/gram, preferably from 4 to 8 meq/gram, more preferably from 5 to 7 meq/gram; (b) at least one anionic surfactant selected from amino acid anionic surfactants; and (c) an amphoteric or non-ionic surfactant or combination thereof, wherein the amount of (b) is from 1 to 30 wt %, preferably from 2 to 25 wt %, more preferably from 3 to 20 wt %, even more preferably from 5 to 15 wt %, based on the weight of the styling shampoo compositions.

5. The styling shampoo compositions according to any one of embodiments 1 to 4, wherein the cationic polymers include copolymers of vinyl monomers having cationic amino or quaternary ammonium functionalities with water soluble spacer monomers such as N-vinylpyrrolidone, preferably those cationic polymers derived from quaternary ammonium monomers, such as vinyl quaternary ammonium monomers having cyclic cationic nitrogen-containing rings, such as imidazolium, for example alkyl vinyl imidazolium, more preferably the compounds referred to in accordance with INCI as Polyquaternium, in particular Polyquaternium-1 to Polyquaternium-74, especially Polyquaternium-16.

6. The styling shampoo compositions according to any one of embodiments 1 to 5, wherein the weight average molecular weight (Mw) of the cationic polymers is from 10,000 to 5,000,000, preferably from 20,000 to 1,000,000, more preferably from 30,000 to 600,000.

7. The styling shampoo compositions according to any one of embodiments 1 to 2 and 5 to 6, wherein the sulfate anionic surfactants include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, and combinations thereof, preferably ammonium laureth sulfate, triethylamine laureth sulfate, triethanolamine laureth sulfate, monoethanolamine laureth sulfate, diethanolamine laureth sulfate, sodium laureth sulfate, potassium laureth sulfate, more preferably sodium laureth sulfate.

8. The styling shampoo compositions according to any one of embodiments 1 and 3 to 6, wherein the amino acid anionic surfactants include acyl glutamate salts, acyl taurate salts, acyl glycinate salts, acyl alaninate salts, acyl sarcosinate salts and acyl aspartate salts.

9. The styling shampoo compositions according to embodiment 8, wherein the acyl glutamate salts include cocoyl glutamate, lauroyl glutamate, myristoyl glutamate, palmitoyl glutamate, stearoyl glutamate, hydrogenated tallow acyl glutamate, olive oil acyl glutamate and octanoyl glutamate, preferably cocoyl glutamate, sodium lauroyl glutamate, sodium myristoyl glutamate, sodium palmitoyl glutamate, sodium stearoyl glutamate, disodium cocoyl glutamate, disodium stearoyl glutamate, potassium cocoyl glutamate, potassium lauroyl glutamate, and potassium myristoyl glutamate, more preferably sodium cocoyl glutamate.

10. The styling shampoo compositions according to embodiment 8, wherein the acyl taurate salts include cocoyl taurates, cocoyl methyl taurates, lauric taurates, lauroyl methyl taurates, stearoyl methyl taurates, myristoyl methyl taurates, palmitoyl methyl taurates, oleoyl methyl taurates, hexanoyl methyl taurates, and lauroyl methyl beta-alanine taurates, preferably cocoyl taurates, cocoyl methyl taurate, lauric acid taurates, lauroyl taurates and lauroyl methyl taurates, preferably sodium cocoyl taurate, potassium methyl cocoyl taurate, sodium methyl cocoyl taurate, magnesium methyl cocoyl taurate, sodium taurine cocoyl methyl taurate, potassium taurine laurate, sodium lauroyl taurate and sodium methyl lauroyl taurate, more preferably sodium taurine cocoyl methyl taurate.

11. The styling shampoo compositions according to embodiment 8, wherein the acyl glycinate salts include cocoyl glycinate, palmitoyl glycinate, octanoyl glycinate and undecylenoyl glycinate, preferably cocoyl glycinate, more preferably potassium cocoyl glycinate and sodium cocoyl glycinate, most preferably sodium cocoyl glycinate.

12. The styling shampoo compositions according to embodiment 8, wherein the acyl alaninate salts include cocoyl alaninate, cocoyl methyl alaninate, lauroyl methyl alaninate, and myristoyl methyl alaninate, preferably cocoyl alaninate, cocoyl methyl alaninate and lauroyl methyl alaninate, more preferably sodium cocoyl alaninate, TEA cocoyl alaninate, sodium cocoyl methyl alaninate, sodium lauroyl methyl alaninate and TEA lauroyl methyl alaninate, most preferably sodium cocoyl alaninate.

13. The styling shampoo compositions according to embodiment 8, wherein the acyl sarcosinate salts include cocoyl sarcosinate, lauroyl sarcosinate, myristoyl sarcosinate, palmitoyl sarcosinate, and oleoyl sarcosinate, preferably cocoyl sarcosinate and lauroyl sarcosinate, more preferably potassium cocoyl sarcosinate, sodium cocoyl sarcosinate, TEA cocoyl sarcosinate, potassium lauroyl sarcosinate, sodium lauroyl sarcosinate and TEA lauroyl sarcosinate, most preferably sodium cocoyl sarcosinate.

14. The styling shampoo compositions according to embodiment 8, wherein the acyl aspartate salts include palmitoyl aspartate, myristyl aspartate, lauryl aspartate, and lauroyl aspartate, preferably lauryl aspartate and lauroyl aspartate, more preferably potassium lauryl aspartate, sodium lauryl aspartate, TEA lauryl aspartate and sodium lauroyl aspartate, most preferably sodium lauroyl aspartate.

15. The styling shampoo compositions according to any one of embodiments 1 to 14, wherein the composition further comprise alkoxylated carboxylates in an amount of from 0.1 to 3% by weight, preferably from 0.5 to 2.5% by weight, more preferably from 1 to 2% by weight, based on the total weight of the composition.

16. The styling shampoo compositions according to embodiment 15, wherein the alkoxylated carboxylates are selected from the group consisting of Trideceth-7 Carboxylic Acid, Sodium Laureth-13 Carboxylates, Sodium Laureth-4 Carboxylates, Laureth-11 Carboxylic Acid, Laureth-5 Carboxylic Acid, Sodium Laureth-5 Carboxylates, Ammonium Laureth-6 Carboxylates, Ammonium Laureth-8 Carboxylates, Capryleth-4 Carboxylic Acid, Capryleth-6 Carboxylic Acid, Capryleth-9 Carboxylic Acid, Ceteareth-25 Carboxylic Acid, Cetyl C12-15 Pareth-8 Carboxylates, Cetyl C12-15 Pareth-9 Carboxylates, Cetyl PPG-2 Isodeceth-7 Carboxylates, Coceth-7 Carboxylic Acid, C9-11 Pareth-6 Carboxylic Acid, C9-11 Pareth-8 Carboxylic Acid, C11-15 Pareth-7 Carboxylic Acid, C12-13 Pareth-5 Carboxylic Acid, C12-13 Pareth-7 Carboxylic Acid, C12-13 Pareth-8 Carboxylic Acid, C12-13 Pareth-12 Carboxylic Acid, C12-15 Pareth-7 Carboxylic Acid, C12-15 Pareth-8 Carboxylic Acid, C12-15 Pareth-12 Carboxylic Acid, C14-15 Pareth-8 Carboxylic Acid, Deceth-7 Carboxylic Acid, Ethylhexeth-3 Carboxylic Acid, Hexeth-4 Carboxylic Acid, Isopropyl C12-15-Pareth-9 Carboxylate, Isopropyl PPG-2 Isodeceth-7 Carboxylates, Isosteareth-6 Carboxylic Acid, Isosteareth-11 Carboxylic Acid, Laureth-3 Carboxylic Acid, Laureth-4 Carboxylic Acid, Laureth-5 Carboxylic Acid, Laureth-6 Carboxylic Acid, Laureth-8 Carboxylic Acid, Laureth-10 Carboxylic Acid, Laureth-11 Carboxylic Acid, Laureth-12 Carboxylic Acid, Laureth-13 Carboxylic Acid, Laureth-14 Carboxylic Acid, Laureth-17 Carboxylic Acid, Magnesium Laureth-11 Carboxylates, MEA-Laureth-6 Carboxylates, MEA PPG-6 Laureth-7 Carboxylates, MEA-PPG-8-Steareth-7 Carboxylates, Myreth-3 Carboxylic Acid, Myreth-5 Carboxylic Acid, Oleth-3 Carboxylic Acid, Oleth-6 Carboxylic Acid, Oleth-10 Carboxylic Acid, PEG-2 Stearamides Carboxylic Acid, PEG-9 Stearamide Carboxylic Acid, Potassium Laureth-3 Carboxylate, Potassium Laureth-4 Carboxylate, Potassium Laureth-5 Carboxylate, Potassium Laureth-6 Carboxylate, Potassium Laureth-10 Carboxylate, Potassium Trideceth-3 Carboxylate, Potassium Trideceth-4 Carboxylate, Potassium Trideceth-7 Carboxylate, Potassium Trideceth-15 Carboxylate, Potassium Trideceth-19 Carboxylate, PPG-3-Deceth-2 Carboxylic Acid, Propyl C12-15 Pareth-8 Carboxylate, Sodium Capryleth-2 Carboxylate, Sodium Capryleth-9 Carboxylate, Sodium Ceteareth-13 Carboxylate, Sodium Ceteth-13 Carboxylate, Sodium C9-11 Pareth-6 Carboxylates, Sodium C11-15 Pareth-7 Carboxylates, Sodium C12-13 Pareth-5 Carboxylates, Sodium C12-13 Pareth-8 Carboxylates, Sodium C12-13 Pareth-12 Carboxylates, Sodium C12-15 Pareth-6 Carboxylates, Sodium C12-15 Pareth-7 Carboxylates, Sodium C12-15 Pareth-8 Carboxylates, Sodium C12-15 Pareth-12 Carboxylates, Sodium C14-15 Pareth-8 Carboxylates, Sodium C12-14 Sec-Pareth-8 Carboxylates, Sodium Deceth-2 Carboxylates, Sodium Hexeth-4 Carboxylates, Sodium Isosteareth-6 Carboxylates, Sodium Isosteareth-11 Carboxylates, Sodium Laureth-3 Carboxylates, Sodium Laureth-4 Carboxylates, Sodium Laureth-5 Carboxylates, Sodium Laureth-6 Carboxylates, Sodium Laureth-8 Carboxylates, Sodium Laureth-11 Carboxylates, Sodium Laureth-12 Carboxylates, Sodium Laureth-13 Carboxylates, Sodium Laureth-14 Carboxylates, Sodium Laureth-16 Carboxylates, Sodium Laureth-17 Carboxylates, Sodium Lauryl Glucose Carboxylates, Sodium Lauryl Glycol Carboxylate, Sodium PEG-6 Cocamide Carboxylate, Sodium PEG-8 Cocamide Carboxylate, Sodium PEG-3 Lauramide Carboxylate, Sodium PEG-4 Lauramide Carboxylate, Sodium PEG-7 Olive Oil Carboxylate, Sodium PEG-8 Palm Glycerides Carboxylate, Sodium Trideceth-3 Carboxylates, Sodium Trideceth-4 Carboxylates, Sodium Trideceth-6 Carboxylates, Sodium Trideceth-7 Carboxylates, Sodium Trideceth-8 Carboxylates, Sodium Trideceth-12 Carboxylates, Sodium Trideceth-15 Carboxylates, Sodium Trideceth-19 Carboxylates, Sodium Undeceth-5 Carboxylates, Trideceth-3 Carboxylic Acid, Trideceth-4 Carboxylic Acid, Trideceth-7 Carboxylic Acid, Trideceth-8 Carboxylic Acid, Trideceth-15 Carboxylic Acid, Trideceth-19 Carboxylic Acid and undeceth-5 Carboxylic acid, and combination thereof, preferably Sodium Lauryl Glucose Carboxylates, Sodium Lauryl Glycol Carboxylate, more preferably Sodium Lauryl Glucose Carboxylates.

17. The styling shampoo compositions according to any one of embodiments 1 to 16, wherein the amphoteric surfactants include sodium acyl amphoacetate, disodium acyl amphodipropionate, disodium alkyl amphodiacetate, sodium acyl am-phohydroxypropylsulfonate, disodium acyl amphodiacetate, sodium acyl amphopropionate, and N-coconut fatty acid amidoethyl N-hydroxyethylglycinate sodium salts; N-alkylamino acids, for example aminopropylalkylglutamide, alkylaminopropionic acid, sodium alkylimidodipropionate and lauroamphocarboxyglycinate; alkylbetaines, alkylamidopropylbetaines, alkylsulfobetaines, alkyl glycinates, alkylcarboxyglycinates, alkyl amphoacetates or -propionates, alkyl amphodiacetates or -dipropionates, preferably cocodimethylsulfopropylbetaine, laurylbetaine, cocoamidopropylbetaine or sodium co-camphopropionate, more preferably cocoamidopropylbetaine.

18. The styling shampoo compositions according to any one of embodiments 1 to 17, wherein the nonionic surfactants include esters which are formed by esterification of carboxylic acids with ethylene oxide, glycerol, sorbitan or other alcohols, ethers, for example ethoxylated alcohols, ethoxylated lanolin, ethoxylated polysiloxanes, propoxylated POE ethers, alkyl polyglycosides, preferably caprylyl/capryl glucoside, lauryl glucoside, decyl glycoside and coco-glycoside, glycosides with an HLB value of at least 20, more preferably coco-glycoside.

19. The styling shampoo compositions according to any one of embodiments 1 to 18, wherein the amount of the amphoteric or non-ionic surfactants is from 0 to 20 wt %, preferably from 2 to 19 wt %, more preferably from 4 to 15 wt %, based on the weight of the styling shampoo compositions.

20. The styling shampoo compositions according to any one of embodiments 1 to 19, wherein the compositions further comprise thickeners.

21. The styling shampoo compositions according to embodiment 20, wherein the thickeners include crosslinked polyacrylic acids and derivatives thereof, polysaccharides, such as xanthan gum, guar guar, agar agar, alginates or tyloses, cellulose derivatives, for example carboxymethylcellulose or hydroxycarboxymethylcellulose, and also relatively high molecular weight polyethylene glycol mono- and diesters of fatty acids, fatty alcohols, monoglycerides and fatty acids, polyvinyl alcohol and polyvinylpyrrolidone, alkanolamides, such as cocamide MEA, cocamide DEA, or cocamide MIPA.

22. The styling shampoo compositions according to any one of embodiments 1 to 21, wherein the compositions further comprise stabilizing agents.

23. The styling shampoo compositions according to embodiment 22, wherein the stabilizing agents include hydroxyalkylcellulose, wherein alkyl is a C₁-C₄-alkyl, particularly hydroxyethylcellulose, starches, acrylate homopolymers or acrylate copolymers, especially acrylate copolymers.

24. The styling shampoo compositions according to any one of embodiments 1 to 23, wherein the pH of the composition is from 4.5 to 7, preferably from 4.8 to 6.9, more preferably from 5.0 to 6.7.

25. A process for preparing the styling shampoo compositions as defined in any one of embodiments 1 to 24, which comprises mixing the components of the compositions in the following order: (a) dissolving the cationic polymer in water; (b) adding the optional non-ionic or amphoteric surfactants; (c) adding the anionic surfactants; (d) adding other components.

26. Use of the styling shampoo compositions as defined in any one of embodiments 1 to 24 for cleaning and styling of human hair.

It has been found that styling shampoo compositions which comprise a cationic polymer of high charge density and at least one anionic surfactant can provide improved styling performance, especially when used in surfactant matrices such as an amphoteric or non-ionic surfactant or combination thereof.

### DESCRIPTION OF THE DRAWING

FIG. 1 provides a comparison for foaming of Ex. 2 vs SET Wet Studio X.

FIG. 2 provides a comparison for styling of Ex. 2 (a) vs SET Wet Studio X (b) after drying.

FIG. 3 shows styling of EX. 2 after drying and breaking.

### DETAILED DESCRIPTION OF THE INVENTION

One aspect of the present invention relates to styling shampoo compositions which comprise (a) a cationic polymer with charge density of from 2 to 10 meq/gram, preferably from 4 to 8 meq/gram, more preferably from 5 to 7 meq/gram; (b) at least one anionic surfactant selected from sulfate anionic surfactants and amino acid anionic surfactants; and (c) optionally an amphoteric or non-ionic surfactant or combination thereof, wherein the amount of (a) is from 0.3 to 10 wt %, preferably from 0.5 to 9 wt %, more preferably from 1 to 8 wt %, even more preferably from 1.5 to 8 wt %, based on the weight of the styling shampoo compositions.

Another aspect of the present invention relates to the process for preparing the styling shampoo compositions, which comprises following steps: (a) dissolving the cationic polymer in water; (b) adding the optional non-ionic or amphoteric surfactants; (c) adding the anionic surfactants; (d) adding other components.

Yet another aspect of the present invention relates to the use of the styling shampoo compositions for cleaning and styling of human hair.

Unless defined otherwise, all technical and scientific terms used herein have the same meaning as commonly understood by one of ordinary skill in the art to which this invention belongs.

Expressions “a”, “an”, “the”, when used to define a term, include both the plural and singular forms of the term.

All percentages, parts and ratios are by weight of the total composition, unless otherwise specified. All such weights as they pertain to listed components are based on the specific ingredient level and, therefore, do not include carriers or by-products that may be included in commercially available materials, unless otherwise specified.

Cationic Polymer

The styling shampoo compositions of the present invention comprise an organic cationic polymer suitable for application to human hair or skin.

The organic cationic polymers useful herein have an open chain backbone which contains quaternary ammonium or cationic amino moieties, or combinations thereof.

As used herein the “charge density” of the cationic polymers is defined as the number of cationic sites per polymer gram atomic weight (molecular weight), and can be expressed in terms of meq/gram of cationic charge.

The weight average molecular weight (Mw) of the cationic polymers is from 10,000 to 5,000,000, preferably from 20,000 to 1,000,000, more preferably from 30,000 to 600,000, preferably measured by light scattering method.

Non-limiting examples of suitable cationic polymers include copolymers of vinyl monomers having cationic amino or quaternary ammonium functionalities with water soluble spacer monomers such as N-vinylpyrrolidone.

Other suitable cationic polymers include those cationic polymers derived from quaternary ammonium monomers such as vinyl quaternary ammonium monomers having cyclic cationic nitrogen-containing rings such as imidazolium, for example alkyl vinyl imidazolium. The alkyl portions of these monomers are preferably lower alkyls such as the C₁-C₃ alkyls, more preferably C₁ and C₂ alkyls.

Preferred cationic polymers include the compounds referred to in accordance with INCI as Polyquaternium, in particular Polyquaternium-1 to Polyquaternium-74, especially those listed in Table 1.

TABLE 1

| INCI | |
|---|---|
| Name | |
| Poly- | |
| quater- | |
| nium | |
| −X | Technical Name |
| −2 | Bis(2-chloroethyl)ether, polym. w. N,N′-bis[3- |
| | (dimethylamino)propyl]urea |
| −4 | Hydroxyethylcellulose Dimethyldiallylammoinum |
| | Chloride Copolymer |
| −5 | Copolymer of acrylamide and beta-methacrylyloxyethyl |
| | trirnethyl ammonium methosulfate |
| −6 | Polydimethyldiallyl Ammonium Chloride |
| −7 | Dimethyldiallyl Ammonium Chloride & Acrylamide |
| | Copolymer |
| −9 | Polydimethyaminoethyl methacrylate quaternized with |
| | Methyl Bromide |
| −10 | Hydroxyethylcellulose reacted with trimethyl ammonium |
| | substituted epoxide |
| −11 | PVP N,N-Dimethyl Aminoethyl Methaerylic Acid |
| | Copolymer Diethyl Sulfate Soln |
| −14 | Ethanaminium, N,N,N-Trimethyl-2-[(2-methyl-1-oxo- |
| | 2-propeny)oxy]-, Methyl Sulfate Homopolymer |
| −15 | Acrylamide-Dimethylaminoethyl Methacrylate Methyl |
| | Chloride Copolymer |
| −16 | 3-Methyl-1-Vinylimidazolium Chloride-1-Vinyl-2- |
| | Pyrrolidinone Chloride |
| −17 | Quat salt made from Adipic acid & diethy/ |
| | aminopropylamine & dichloroether |
| −18 | Quat salt prepared by the reaction of adipic acid and |
| | dimethylaminopropylamine, reacted with dichloroethyl ether |
| −19 | Quat ammonium salt prepared by the reaction of |
| | polyvinyl alcohol with 2,3-epoxypropylamine |
| −20 | Quat ammonium salt prepared by the reaction of polyvinyl |
| | octadecyl ether with 2,3-epoxypropylamine |
| −22 | Acrylic Acid-Diallyldimethylammonium Chloride |
| | (DADMAC) Polymer |
| −24 | Polyquat ammonium salt of hydroxyethyl cellulose reacted |
| | with lauryl dimethyl ammonium substituted epoxide |
| −27 | Block Copolymer of Polyquaternium-2 and 17 |
| −28 | Vinylpyrrolidone/Methacrylamidopropyltrimethylammonium |
| | Chloride Copolymer |
| −29 | Propoxylated Chitosan quaternized with epichlorhydrin |
| −30 | Ethanaminium, N-Carboxymethyl)-N, N-Dimethyl-2- |
| | ((2-Methyl-1-oxo-2-Propenyl)Oxy)-, Inner Salt, Polymer |
| | with Methyl 2-Methyl-2-Propenoate |
| −31 | 2-propane nitrile reaction product w/N, N-dimethylpropane- |
| | diamine, Sulfate |
| −32 | Acrylamide-Dimethylaminoethyl Methacrylate Methyl |
| | Chloride (DMAEMA) Copolymer |
| −37 | Trimethylaminoethyl Methacrylate Chloride Polymer |
| −39 | Acrylic Acid (AA), Polymer w/Acrylamide & |
| | Diallyldimethylammonium Chloride(DADMAC) |
| −42 | Polyoxyethylene (dimethyliminio)ethylene-(dimethyliminio) |
| | ethylene dichloride |
| −43 | Copolymer of Acrylamide, acrylamidopropyltrimonium |
| | chloride, amidopropylacrylamide & DMAPA Monomers |
| | monomers |
| −44 | Polyquat ammonium salt of vinylpyrrilidone & quaternized |
| | monomers |
| −46 | Quat ammonium salt of vinylcaprolactum, vinylpyrrolidone |
| | & methylvinylimidazolium |
| −47 | Quat ammonium chloride- acrylic acid, methyl acrylate & |
| | methacrylamidopropyltrimonium Chloride |
| −48 | Copolymer of methacryolyl ethyl betaine, 2-hydroxyethyl- |
| | methacrylate & methacryloylethyltrimethylammonium |
| | chloride |
| −51 | 3,5,8-Triox-4-Phosphaundec-10-en-1-aminium, 4- |
| | Hydroxy-N,N,N, 10-Tetramethyl-9-Oxo, Inner Salt, 4- |
| | Oxide, Polymer with Butyl 2-Methyl-2-Propenoate |
| −53 | Acrylic Acid (AA)/Acrylamide/Methacrylamido- |
| | propyltrimonium Chloride (MAPTAC) Copolymer |
| −54 | Polymeric quaternary ammonium salt prepared by the |
| | reaction of aspartic acid and C6-18 alkylamine with |
| | dimethylaminopropylamine and sodium chloroacetate |
| −55 | 1-Dodecanaminium, N,N-Dimethyl-N-[3-[(2-Methyl-1- |
| | Oxo-2-Propenyl)AminoPropyl]-, Chloride, Polymer with |
| | N-[3-(Dimethylamino) Propyl]-2-Methyl-2- |
| | Propenamide and 1-Ethenyl-2-Pyrrolidinone |
| −56 | Polymeric quaternary ammonium salt prepared by the |
| | reaction of aspartic acid and C6-18 alkylamine with |
| | dimethylaminopropylamine and sodium chloroacetate. |
| −57 | Polymeric quaternary ammonium salt consisting of Castor |
| | Isostearate Succinate (q.v.) and Ricinoleamidopropyl- |
| | trimonium Chloride (q.v.) monomers |
| −58 | 2-Propenoic Acid, Methyl Ester, Polymer with 2,2-Bis[(2- |
| | Propenyloxy) Methyl]-1-Butanol and Diethenylbenzene, |
| | Reaction Products with N,N-Dimethyl-1,3- |
| | Propanediamine, Chloromethane-Quaternized |
| −59 | Polyquatemium polyester |
| −60 | 9-Octadecenoic Acid, 12-Hydroxy-, [(2-Hydroxyethyl) |
| | Imino]Di-2, 1-Ethanediyl Ester, Polymer with 5- |
| | Isocyanato-1-(Isocyanatomethyl)-1,3,3-Trimethyl |
| | cyclohexane, Compd. with Diethyl Sulfate |
| −62 | Polymeric quaternary ammonium salt prepared by the |
| | reaction of butyl methacrylate, polyethylene glycol |
| | methyl ether methacrylate, ethylene glycol dimethacrylate |
| | and 2-methacryloyethyl trimoniurn chloride with |
| | 2,2′-azobis(2-methyl propionamidine) dihydrochloride |
| −63 | Copolymer of acrylamide, acrylic acid and |
| | ethyltrimonium chloride acrylate |
| −65 | Polymeric quaternary ammonium salt consisting of 2- |
| | methacryloyloxyethylphosphorylcholine, butyl |
| | methacrylate and sodium methacrylate monomers |
| −68 | Quatemized copolymers of vinylpyrrolidone (VP), |
| | methacrylamide(MAM) vinylimidazole(VI) & |
| | quaternized vinylimidazole (QVI) |
| −69 | Polymeric quaternary ammonium salt containing |
| | vinyl caprolactam, vinylpyrradone, dimethylaminopropyl |
| | methacrylamide (DMAPA), and |
| | methoacryloylaminopropyl lauryldimonium chloride |

In one preferred embodiment, Polyquaternium includes Polyquaternium-2, Polyquaternium-4, Polyquaternium-5, Polyquaternium-6, Polyquaternium-7, Polyquaternium-10, Polyquaternium-11, Polyquaternium-16, Polyquaternium-22, Polyquaternium-24, Polyquaternium-28, Polyquaternium-32, Polyquaternium-37, Polyquaternium-39, Polyquaternium-42, Polyquaternium-43, Polyquaternium-44, Polyquaternium-46, Polyquaternium-47, Polyquaternium-51, Polyquaternium-53, Polyquaternium-55, Polyquaternium-57, Polyquaternium-58, Polyquaternium-59, Polyquaternium-60, Polyquaternium-63, Polyquaternium-64, Polyquaternium-65, Polyquaternium-68, and combinations thereof.

In another preferred embodiment, Polyquaternium includes Polyquaternium-2, Polyquaternium-4, Polyquaternium-6, Polyquaternium-7, Polyquaternium-11, Polyquaternium-16, Polyquaternium-22, Polyquaternium-28, Polyquaternium-32, Polyquaternium-37, Polyquaternium-39, Polyquaternium-42, Polyquaternium-47, Polyquaternium-51, Polyquaternium-53, Polyquaternium-55, Polyquaternium-58, and combinations thereof.

In another preferred embodiment, Polyquaternium includes Polyquaternium-16, such as those commercially available from BASF under Luviquat tradename (for example Luviquat® Excellence, Luviquat® HM 552, Luviquat® FC 370, Luviquat® FC 550, Luviquat® FC 905). The most preferred cationic polymer is Luviquat® Excellence (BASF).

Preferably, the amount of cationic polymer is from 0.3 to 10 wt %, preferably from 0.5 to 9 wt %, more preferably from 1 to 8 wt %, even more preferably from 1.5 to 8 wt %, based on the weight of the styling shampoo compositions.

Anionic Surfactant

Suitable anionic surfactants for use in the styling shampoo composition herein include sulfate anionic surfactants and amino acid anionic surfactants.

Non-limiting examples of suitable sulfate anionic surfactants include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, and combinations thereof, preferably ammonium laureth sulfate, triethylamine laureth sulfate, triethanolamine laureth sulfate, monoethanolamine laureth sulfate, diethanolamine laureth sulfate, sodium laureth sulfate, potassium laureth sulfate, more preferably sodium laureth sulfate (for example Texapon® NSO UP (BASF), Texapon® OC-P (BASF), Texapon® OC-N (BASF), Texapon® N 703 (BASF), Texapon® N 701 (BASF), Texapon® N 70 (BASF), and Texapon® N 28 S (BASF)).

Non-limiting examples of suitable amino acid anionic surfactants include acyl glutamate salts, acyl taurate salts, acyl glycinate salts, acyl alaninate salts, acyl sarcosinate salts and acyl aspartate salts. Examples of the salts include alkali metal salts such as sodium salt (Na) and potassium salt (K); alkaline earth metal salts, such as calcium (Ca) and magnesium (Mg) salts; triethanolamine salts (TEA); an ammonium salt; and so on. More preferred are, for example, potassium salts, sodium salts, triethanolamine salts and ammonium salts, in particular sodium salts.

Preferably, the acyl glutamate salts include cocoyl glutamate, lauroyl glutamate, myristoyl glutamate, palmitoyl glutamate, stearoyl glutamate, hydrogenated tallow acyl glutamate, olive oil acyl glutamate and octanoyl glutamate, for example, sodium cocoyl glutamate, sodium lauroyl glutamate, sodium myristoyl glutamate, sodium palmitoyl glutamate, sodium stearoyl glutamate, disodium cocoyl glutamate, disodium stearoyl glutamate, potassium cocoyl glutamate, potassium lauroyl glutamate, and potassium myristoyl glutamate, more preferably sodium cocoyl glutamate (for example Plantapon® ACG-LC (BASF), Plantapon® ACG HC (BASF), Plantapon® ACG 35 (BASF), Plantapon® ACG 50 (BASF), Hostapon® KCG (Clariant), and Hostapon® KCG (Clariant)).

Preferably, the acyl taurate salts include cocoyl taurates, cocoyl methyl taurates, lauric taurates, lauroyl methyl taurates, stearoyl methyl taurates, myristoyl methyl taurates, palmitoyl methyl taurates, oleoyl methyl taurates, hexanoyl methyl taurates, and lauroyl methyl beta-alanine taurates. Particularly preferred are, for example, cocoyl taurates, cocoyl methyl taurate, lauric acid taurates, lauroyl taurates and lauroyl methyl taurates. Further particularly preferred are, for example, sodium cocoyl taurate, potassium methyl cocoyl taurate, sodium methyl cocoyl taurate, magnesium methyl cocoyl taurate, sodium taurine cocoyl methyl taurate, potassium taurine laurate, sodium lauroyl taurate and sodium methyl lauroyl taurate, more preferably sodium taurine cocoyl methyl taurate (for example NEOSCOAP CDT-30 (CR), NEOSCOAPCDT-30-SF(CR), NEOSCOAP CDT-30-SF, NEOSCOAP CN-30(CR), and NEOSCOAP CN-30-SF (Toho Chemical Industry)).

Preferably, the acyl glycinate salts include cocoyl glycinate, palmitoyl glycinate, octanoyl glycinate and undecylenoyl glycinate. Even more preferred are, for example, cocoyl glycinate. Particularly preferred are, for example, potassium cocoyl glycinate and sodium cocoyl glycinate, more preferably sodium cocoyl glycinate.

Preferably, the acyl alaninate salts include cocoyl alaninate, cocoyl methyl alaninate, lauroyl methyl alaninate, and myristoyl methyl alaninate. Even more preferred are, for example, cocoyl alaninate, cocoyl methyl alaninate and lauroyl methyl alaninate. Particularly preferred are, for example, sodium cocoyl alaninate, TEA cocoyl alaninate, sodium cocoyl methyl alaninate, sodium lauroyl methyl alaninate and TEA lauroyl methyl alaninate, more preferably sodium cocoyl alaninate.

Preferably, the acyl sarcosinate salts include cocoyl sarcosinate, lauroyl sarcosinate, myristoyl sarcosinate, palmitoyl sarcosinate, and oleoyl sarcosinate. Particularly preferred are, for example, cocoyl sarcosinate and lauroyl sarcosinate. Further particularly preferred are, for example, potassium cocoyl sarcosinate, sodium cocoyl sarcosinate, TEA cocoyl sarcosinate, potassium lauroyl sarcosinate, sodium lauroyl sarcosinate and TEA lauroyl sarcosinate, more preferably sodium cocoyl sarcosinate.

Preferably, the acyl aspartate salts include palmitoyl aspartate, myristyl aspartate, lauryl aspartate, and lauroyl aspartate. Even more preferred are, for example, lauryl aspartate and lauroyl aspartate. Particularly preferred are potassium lauryl aspartate, sodium lauryl aspartate, TEA lauryl aspartate and sodium lauroyl aspartate, more preferably sodium lauroyl aspartate.

In one preferred embodiment, the composition according to the invention can further comprise alkoxylated carboxylates in an amount of from 0.1 to 3% by weight, preferably from 0.5 to 2.5% by weight, more preferably from 1 to 2% by weight, based on the total weight of the composition, which are selected from the group consisting of Trideceth-7 Carboxylic Acid, Sodium Laureth-13 Carboxylates, Sodium Laureth-4 Carboxylates, Laureth-11 Carboxylic Acid, Laureth-5 Carboxylic Acid, Sodium Laureth-5 Carboxylates, Ammonium Laureth-6 Carboxylates, Ammonium Laureth-8 Carboxylates, Capryleth-4 Carboxylic Acid, Capryleth-6 Carboxylic Acid, Capryleth-9 Carboxylic Acid, Ceteareth-25 Carboxylic Acid, Cetyl C12-15 Pareth-8 Carboxylates, Cetyl C12-15 Pareth-9 Carboxylates, Cetyl PPG-2 Isodeceth-7 Carboxylates, Coceth-7 Carboxylic Acid, C9-11 Pareth-6 Carboxylic Acid, C9-11 Pareth-8 Carboxylic Acid, C11-15 Pareth-7 Carboxylic Acid, C12-13 Pareth-5 Carboxylic Acid, C12-13 Pareth-7 Carboxylic Acid, C12-13 Pareth-8 Carboxylic Acid, C12-13 Pareth-12 Carboxylic Acid, C12-15 Pareth-7 Carboxylic Acid, C12-15 Pareth-8 Carboxylic Acid, C12-15 Pareth-12 Carboxylic Acid, C14-15 Pareth-8 Carboxylic Acid, Deceth-7 Carboxylic Acid, Ethylhexeth-3 Carboxylic Acid, Hexeth-4 Carboxylic Acid, Isopropyl C12-15-Pareth-9 Carboxylate, Isopropyl PPG-2 Isodeceth-7 Carboxylates, Isosteareth-6 Carboxylic Acid, Isosteareth-11 Carboxylic Acid, Laureth-3 Carboxylic Acid, Laureth-4 Carboxylic Acid, Laureth-5 Carboxylic Acid, Laureth-6 Carboxylic Acid, Laureth-8 Carboxylic Acid, Laureth-10 Carboxylic Acid, Laureth-11 Carboxylic Acid, Laureth-12 Carboxylic Acid, Laureth-13 Carboxylic Acid, Laureth-14 Carboxylic Acid, Laureth-17 Carboxylic Acid, Magnesium Laureth-11 Carboxylates, MEA-Laureth-6 Carboxylates, MEA PPG-6 Laureth-7 Carboxylates, MEA-PPG-8-Steareth-7 Carboxylates, Myreth-3 Carboxylic Acid, Myreth-5 Carboxylic Acid, Oleth-3 Carboxylic Acid, Oleth-6 Carboxylic Acid, Oleth-10 Carboxylic Acid, PEG-2 Stearamides Carboxylic Acid, PEG-9 Stearamide Carboxylic Acid, Potassium Laureth-3 Carboxylate, Potassium Laureth-4 Carboxylate, Potassium Laureth-5 Carboxylate, Potassium Laureth-6 Carboxylate, Potassium Laureth-10 Carboxylate, Potassium Trideceth-3 Carboxylate, Potassium Trideceth-4 Carboxylate, Potassium Trideceth-7 Carboxylate, Potassium Trideceth-15 Carboxylate, Potassium Trideceth-19 Carboxylate, PPG-3-Deceth-2 Carboxylic Acid, Propyl C12-15 Pareth-8 Carboxylate, Sodium Capryleth-2 Carboxylate, Sodium Capryleth-9 Carboxylate, Sodium Ceteareth-13 Carboxylate, Sodium Ceteth-13 Carboxylate, Sodium C9-11 Pareth-6 Carboxylates, Sodium C11-15 Pareth-7 Carboxylates, Sodium C12-13 Pareth-5 Carboxylates, Sodium C12-13 Pareth-8 Carboxylates, Sodium C12-13 Pareth-12 Carboxylates, Sodium C12-15 Pareth-6 Carboxylates, Sodium C12-15 Pareth-7 Carboxylates, Sodium C12-15 Pareth-8 Carboxylates, Sodium C12-15 Pareth-12 Carboxylates, Sodium C14-15 Pareth-8 Carboxylates, Sodium C12-14 Sec-Pareth-8 Carboxylates, Sodium Deceth-2 Carboxylates, Sodium Hexeth-4 Carboxylates, Sodium Isosteareth-6 Carboxylates, Sodium Isosteareth-11 Carboxylates, Sodium Laureth-3 Carboxylates, Sodium Laureth-4 Carboxylates, Sodium Laureth-5 Carboxylates, Sodium Laureth-6 Carboxylates, Sodium Laureth-8 Carboxylates, Sodium Laureth-11 Carboxylates, Sodium Laureth-12 Carboxylates, Sodium Laureth-13 Carboxylates, Sodium Laureth-14 Carboxylates, Sodium Laureth-16 Carboxylates, Sodium Laureth-17 Carboxylates, Sodium Lauryl Glucose Carboxylates, Sodium Lauryl Glycol Carboxylate, Sodium PEG-6 Cocamide Carboxylate, Sodium PEG-8 Cocamide Carboxylate, Sodium PEG-3 Lauramide Carboxylate, Sodium PEG-4 Lauramide Carboxylate, Sodium PEG-7 Olive Oil Carboxylate, Sodium PEG-8 Palm Glycerides Carboxylate, Sodium Trideceth-3 Carboxylates, Sodium Trideceth-4 Carboxylates, Sodium Trideceth-6 Carboxylates, Sodium Trideceth-7 Carboxylates, Sodium Trideceth-8 Carboxylates, Sodium Trideceth-12 Carboxylates, Sodium Trideceth-15 Carboxylates, Sodium Trideceth-19 Carboxylates, Sodium Undeceth-5 Carboxylates, Trideceth-3 Carboxylic Acid, Trideceth-4 Carboxylic Acid, Trideceth-7 Carboxylic Acid, Trideceth-8 Carboxylic Acid, Trideceth-15 Carboxylic Acid, Trideceth-19 Carboxylic Acid and undeceth-5 Carboxylic acid, and combination thereof, preferably Sodium Lauryl Glucose Carboxylates, Sodium Lauryl Glycol Carboxylate, more preferably Sodium Lauryl Glucose Carboxylates (for example Plantapon® LGC Sorb (BASF)).

Preferably, the amount of anionic surfactant is from 1 to 30 wt %, preferably from 2 to 25 wt %, more preferably from 3 to 20 wt %, even more preferably from 5 to 15 wt %, based on the weight of the styling shampoo compositions.

Amphoteric or Non-Ionic Surfactant

Non-limiting examples of suitable amphoteric surfactants include sodium acyl amphoacetate, disodium acyl amphodipropionate, disodium alkyl amphodiacetate, sodium acyl amphohydroxypropylsulfonate, disodium acyl amphodiacetate, sodium acyl amphopropionate, and N-coconut fatty acid amidoethyl N-hydroxyethylglycinate sodium salts.

Further advantageous amphoteric surfactants are N-alkylamino acids, for example aminopropylalkylglutamide, alkylaminopropionic acid, sodium alkylimidodipropionate and lauroamphocarboxyglycinate.

Suitable amphoteric surfactants are also, for example, alkylbetaines, alkylamidopropylbetaines, alkylsulfobetaines, alkyl glycinates, alkylcarboxyglycinates, alkyl amphoacetates or -propionates, alkyl amphodiacetates or -dipropionates. For example, cocodimethylsulfopropylbetaine, laurylbetaine, cocoamidopropylbetaine or sodium cocoamphopropionate, for example Dethyton® PK 45 (BASF), Dethyton® KE-AS (BASF), Dethyton® K/15 (BASF), TEGO® Betain P 50 C (Evonik), TEGO® Betain F 50 (Evonik), and Genagen® CAB 818 (Clariant) can be used.

Non-limiting examples of suitable nonionic surfactants include esters which are formed by esterification of carboxylic acids with ethylene oxide, glycerol, sorbitan or other alcohols, ethers, for example ethoxylated alcohols, ethoxylated lanolin, ethoxylated polysiloxanes, propoxylated POE ethers, alkyl polyglycosides, such as caprylyl/capryl glucoside, lauryl glucoside, decyl glycoside and coco-glycoside, glycosides with an HLB value of at least 20, for example Plantacare® 818 UP (BASF), Plantacare® 1200 UP (BASF), and Plantacare® 2000 UP (BASF).

The amount of the amphoteric or non-ionic surfactant is from 0 to 20 wt %, preferably from 2 to 19 wt %, more preferably from 4 to 15 wt %, based on the weight of the styling shampoo compositions.

In other preferred embodiments, the styling shampoo compositions of the present invention further comprise the following other ingredients.

Hair Fixative

Suitable hair fixative may be used. Suitable examples include, for example VP/methacryl amide/vinyl imidazole copolymer (for example LUVISET® Clear AT3 (BASF)), acrylic acid/vinylpyrrolidone crosspolymer (for example Ultrathix™ P-100 (ISP), vinylcaprolactam/VP/dimethylaminoethyl methacrylate copolymer (Advantage® S (ISP)), and VP/VA Copolymer.

Thickeners

Suitable thickeners for the compositions according to the invention are crosslinked polyacrylic acids and derivatives thereof, polysaccharides, such as xanthan gum, guar guar, agar agar, alginates or tyloses, cellulose derivatives, for example carboxymethylcellulose or hydroxycarboxymethylcellulose, and also relatively high molecular weight polyethylene glycol mono- and diesters of fatty acids, fatty alcohols, monoglycerides and fatty acids, polyvinyl alcohol and polyvinylpyrrolidone, alkanolamides, such as cocamide MEA, cocamide DEA, or cocamide MIPA (for example Comperlan® 100 C (BASF), and Comperlan® 100 (BASF)).

Suitable thickeners are also polyacrylates, such as Carbopol® (Noveon), Ultrez® (Noveon), Luvigel® EM (BASF), Capigel®98 (Seppic), Synthalene® (Sigma), the Aculyn® grades from Rohm and Haas, such as Aculyn® 22 (copolymer of acrylates and methacrylic acid ethoxylates with stearyl radical (20 ethylene oxide (EO) units)) and Aculyn® 28 (copolymer of acrylates and methacrylic acid ethoxylates with behenyl radical (25 EO units)).

Suitable thickeners are furthermore, for example, aerosol grades (hydrophilic silicas), polyacrylamides, polyvinyl alcohol and polyvinylpyrrolidone, surfactants, such as, for example, ethoxylated fatty acid glycerides, esters of fatty acids with polyols, such as, for example, pentaerythritol or trimethylolpropane, fatty alcohol ethoxylates with a narrowed homolog distribution or alkyl oligoglucosides, and also electrolytes such as sodium chloride and ammonium chloride.

Particularly in the case of relatively high-concentration shampoo compositions, it is also possible, for regulating the consistency, to add substances which reduce the viscosity of the composition, such as, for example, propylene glycol or glycerol. These substances only influence the product properties slightly.

Preservatives

The compositions according to the invention can advantageously comprise one or more preservatives. Products with high water contents, for example, shampoos, have to be reliably protected against the build-up of germs. The most important preservatives used for this purpose are urea condensates, p-hydroxybenzoic acid esters, the combination of phenoxyethanol with methyldibromoglutaronitrile and acid preservatives containing benzoic acid, salicylic acid and sorbic acid.

Advantageous preservatives within the context of the present invention are, for example, formaldehyde donors (such as, for example, DMDM hydantoin, which is commercially available, for example, under the trade name Glydant® (Lonza)), iodopropyl butylcarbamates (for example Glycacil-L®, Glycacil-S® (Lonza), Dekaben® LMB (Jan Dekker)), parabens (p-hydroxybenzoic acid alkyl esters, such as, for example, methyl, ethyl, propyl and/or butylparaben), dehydroacetic acid (Euxyl® K 702 (SchQlke & Mayr), phenoxyethanol, ethanol, benzoic acid. So-called preservation aids, such as, for example, octoxyglycerol, glycine, soya etc., are also advantageously used.

Also advantageous are preservatives or preservation aids customary in cosmetics, such as dibromodicyanobutane (2-bromo-2-bromomethylglutarodinitrile), phenoxyethanol, 3-iodo-2-propynyl butylcarbamate, 2-bromo-2-nitropropane-1,3-diol, imidazolidinylurea, 5-chloro-2-methyl-4-isothiazolin-3-one, 2-chloroacetamide, benzalconium chloride, benzyl alcohol, salicylic acid and salicylates.

Complexing Agents

Since the raw materials and also the shampoos themselves are prepared predominantly in steel apparatuses, the end products can comprise iron (ions) in trace amounts. In order to prevent these impurities adversely affecting the product quality via reactions with dyes and perfume oil constituents, complexing agents or chelating agents, such as salts of ethylenediaminetetraacetic acid, of nitrilotriacetic acid, of iminodisuccinic acid or phosphates are added.

UV Photoprotective Filters

In order to stabilize the components present in the compositions according to the invention, such as, for example, dyes and perfume oils, against changes as a result of UV light, UV photoprotective filters, such as, for example, benzophenone derivatives, can be incorporated. All cosmetically acceptable UV photoprotective filters are suitable for this purpose.

Antioxidants

A content of antioxidants is generally preferred. According to the invention, antioxidants which can be used are all antioxidants which are customary or suitable for cosmetic applications. The antioxidants are advantageously selected from the group consisting of amino acids (for example glycine, histidine, tyrosine, tryptophan) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example acarotene, μ-carotene, γ-lycopene) and derivatives thereof, chlorogenic acid and derivatives thereof, liponic acid and derivatives thereof (for example dihydrolipoic acid), aurothioglucose, propylthiouracil and other thiols (for example thioredoxin, glutathione, cysteine, cystine, cystamine and the glycosyl, N acetyl, methyl, ethyl, propyl, amyl, butyl and lauryl, palmitoyl, oleyl, γ-linoleyl, cholesteryl and glyceryl esters thereof) and also salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (for example buthionine sulfoximines, homocysteine sulfoximine, buthionine sulfones, penta-, hexa-, heptathionine sulfoximine) in very low tolerated doses (for example pmol to μmol/kg), also (metal) chelating agents (for example α-hydroxy fatty acids, palmitic acid, phytic acid, lactoferrin), α-hydroxy acids (for example citric acid, lactic acid, maleic acid), humic acid, bile acid, bile extracts, bilirubin, biliverdine, EDTA, EGTA and derivatives thereof, unsaturated fatty acids and derivatives thereof (for example γ-linolenic acid, linoleic acid, oleic acid), folic acid and derivatives thereof, furfurylidenesorbitol and derivatives thereof, ubiquinone and ubiquinol and derivatives thereof, vitamin C and derivatives (for example ascorbyl palmitate, Mg ascorbyl phosphate, ascorbyl acetate), tocopherols and derivatives (for example vitamin E acetate), vitamin A and derivatives (vitamin A palmitate), and coniferyl benzoate of benzoin resin, rutinic acid and derivatives thereof, aglycosylrutine, ferulic acid, furfurylideneglucitol, carnosine, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (for example ZnO, ZnSO₄), selenium and derivatives thereof (for example selenomethionine), stilbenes and derivatives thereof (for example stilbene oxide, trans-stilbene oxide) and the derivatives (salts, esters, ethers, sugars, nucleotides, nucleosides, peptides and lipids) suitable according to the invention of these specified active ingredients.

The amount of the abovementioned antioxidants (one or more compounds) in the compositions is from 0.001 to 10% by weight, preferably from 0.01 to 5% by weight, more preferably from 0.05 to 1% by weight, based on the total weight of the composition.

Buffers

Buffers ensure the pH stability of the compositions. By and large, citrate, lactate and phosphate buffers are used.

Solubility Promoters

The solubility promoters are used in order to dissolve care oils or perfume oils to give clear solutions and also to maintain clear solutions at low temperature. The most common solubility promoters are ethoxylated nonionic surfactants, for example hydrogenated and ethoxylated castor oils.

Antimicrobial Agents

Furthermore, antimicrobial agents can also be used. These include, in general, all suitable preservatives with a specific effect toward Gram-positive bacteria, for example triclosan (2,4,4′-trichloro-2′-hydroxydiphenyl ether), chlorhexidine (1,1′-hexamethylenebis[5-(4-chlorophenyl)biguanide), and TTC (3,4,4′-trichlorocarbanilide). Numerous fragrances also have antimicrobial properties. Also, a large number of essential oils or their characteristic ingredients, such as, for example, oil of cloves (eugenol), mint oil (menthol) or thyme oil (thymol), exhibit marked antimicrobial effectiveness.

The antimicrobial agents are generally used in a concentration of from about 0.1 to 0.3% by weight, based on the total weight of the composition.

Dispersants

If insoluble active ingredients, for example antidandruff active ingredients or silicone oils, are to be dispersed or kept permanently in suspension in the compositions according to the invention, dispersants, for example, magnesium aluminum silicates, bentonites, fatty acyl derivatives, polyvinylpyrrolidone or hydrocolloids, for example xanthan gum or carbomers, have to be used.

According to the invention, dispersants in a total concentration of at most 2% by weight, preferably at most 1.5% by weight and particularly preferably at most 1% by weight, based on the total weight of the composition, are present.

Oils, Fats and Waxes

The compositions according to the invention preferably comprise oils, fats and/or waxes.

Constituents of the oil phase and/or fat phase of the composition according to the invention are advantageously selected from the group of lecithins and fatty acid triglycerides, namely the triglycerol esters of saturated and/or unsaturated, branched and/or unbranched alkanecarboxylic acids of chain length from 8 to 24, in particular 12 to 18 carbon atoms. The fatty acid triglycerides can, for example, advantageously be selected from the group of synthetic, semisynthetic and natural oils, such as, for example, olive oil, sunflower oil, soya oil, peanut oil, rapeseed oil, almond oil, palm oil, coconut oil, castor oil, wheat germ oil, grapeseed oil, safflower oil, evening primrose oil, macadamia nut oil and the like. Further polar oil components can be selected from the group of esters of saturated and/or unsaturated, branched and/or unbranched alkanecarboxylic acids of chain length from 3 to 30 carbon atoms with saturated and/or unsaturated, branched and/or unbranched alcohols of chain length from 3 to 30 carbon atoms, and also from the group of esters of aromatic carboxylic acids with saturated and/or unsaturated, branched and/or unbranched alcohols of chain length from 3 to 30 carbon atoms. Such ester oils can then advantageously be selected from the group isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate dicaprylyl carbonate (Cetiol CC) and cocoglycerides (Myritol 331), butylene glycol dicaprylate/dicaprate and dibutyl adipate, and also synthetic, semisynthetic and natural mixtures of such esters, such as, for example, jojoba oil.

Furthermore, one or more oil components can advantageously be selected from the group of branched and unbranched hydrocarbons and hydrocarbon waxes, silicone oils, dialkyl ethers, the group of saturated or unsaturated, branched or unbranched alcohols.

Any desired mixtures of such oil and wax components are also to be used advantageously within the context of the present invention. If appropriate, it may also be advantageous to use waxes, for example cetyl palmitate, as the sole lipid component of the oil phase.

According to the invention, the oil component is advantageously selected from the group 2-ethylhexyl isostearate, octyldodecanol, isotridecyl isononanoate, isoeicosane, 2-ethylhexyl cocoate, C₁₂₋₁₅-alkyl benzoate, capryl-capric acid triglyceride, dicaprylyl ether.

Mixtures of C₁₂₋₁₅-alkyl benzoate and 2-ethylhexyl isostearate, mixtures of C₁₂₋₁₅-allylbenzoate and isotridecyl isononanoate, and also mixtures of C₁₂₋₁₅-allylbenzoate, 2-ethylhexyl isostearate and isotridecyl isononanoate are advantageous according to the invention.

According to the invention, the oils with a polarity of from 5 to 50 mN/m used are particularly preferably fatty acid triglycerides, in particular soya oil and/or almond oil.

Of the hydrocarbons, paraffin oil, squalane, squalene and in particular (if appropriate hydrogenated) polyisobutenes are to be used advantageously within the context of the present invention.

In addition, the oil phase can advantageously be selected from the group of Guerbet alcohols.

Guerbet alcohols preferred according to the invention are 2-butyloctanol (commercially available, for example, as Isofol®12 (Condea)) and 2-hexyldecanol (commercially available, for example as Isofol®16 (Condea)).

Mixtures of Guerbet alcohols according to the invention are also to be used advantageously according to the invention, such as, for example, mixtures of 2-butyloctanol and 2-hexyldecanol (commercially available, for example, as Isofol®14 (Condea)).

Any desired mixtures of such oil and wax components are also to be used advantageously within the context of the present invention.

Among the polyolefins, polydecenes are the preferred substances.

Advantageously, the oil component can also have a content of cyclic or linear silicone oils or consist entirely of such oils, although it is preferred to use an additional content of other oil phase components apart from the silicone oil or the silicone oils.

Low molecular weight silicones or silicone oils are generally defined by the following general formula

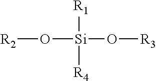

Higher molecular weight silicones or silicone oils are generally defined by the following general formula

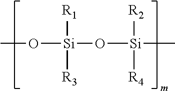

where the silicon atoms may be substituted by identical or different alkyl radicals and/or aryl radicals, which are shown here in general terms by the radicals R₁ to R₄. However, the number of different radicals is not necessarily limited to up to 4. m can here assume values from 2 to 200,000.

Cyclic silicones to be used advantageously according to the invention are generally defined by the following general formula

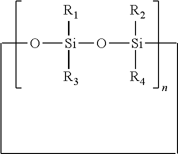

where the silicon atoms may be substituted by identical or different alkyl radicals and/or aryl radicals, which are shown here in general terms by the radicals R₁ to R₄. However, the number of different radicals is not necessarily limited to up to 4. n can here assume values from 3/2 to 20. Fractional values for n take into consideration that odd numbers of siloxyl groups may be present in the cycle.

Phenyltrimethicone is advantageously selected as silicone oil. Other silicone oils, for example dimethicone, hexamethylcyclotrisiloxane, phenyldimethicone, cyclomethicone (for example decamethylcyclopentasiloxane), hexamethylcyclotrisiloxane, polydimethyl-siloxane, poly(methylphenylsiloxane), cetyldimethicone, behenoxydimethicone, are also to be used advantageously within the context of the present invention. Mixtures of cyclomethicone and isotridecyl isononanoate, and also those of cyclomethicone and 2-ethylhexyl isostearate are also advantageous.

However, it is also advantageous to select silicone oils of similar constitution to that of the abovementioned compounds whose organic side chains are derivatized, for example polyethoxylated and/or polypropoxylated. These include, for example, polysiloxane polyalkyl-polyether copolymers, such as, for example, cetyl-dimethicone copolyol.

Cyclomethicone (octamethylcyclotetrasiloxane) is advantageously used as silicone oil to be used according to the invention.

Fat and/or wax components to be used advantageously according to the invention can be selected from the group of vegetable waxes, animal waxes, mineral waxes and petrochemical waxes. For example, candelilla wax, carnauba wax, Japan wax, esparto grass wax, cork wax, guaruma wax, rice germ oil wax, sugarcane wax, berry wax, ouricury wax, montan wax, jojoba wax, shea butter, beeswax, shellac wax, spermaceti, lanolin (wool wax), uropygial grease, ceresine, ozokerite (earth wax), paraffin waxes and microwaxes are advantageous.

Further advantageous fat and/or wax components are chemically modified waxes and synthetic waxes, such as, for example, Syncrowax® HRC (glyceryl tribehenate), and Syncrowax® AW 1 C (C₁₈₋₃₆-fatty acid), and also montan ester waxes, sasol waxes, hydrogenated jojoba waxes, synthetic or modified beeswaxes (for example dimethicone copolyol beeswax and/or C₃₀₋₅₀-alkyl beeswax), cetyl ricinoleates, such as, for example, Tegosoft® CR, polyalkylene waxes, polyethylene glycol waxes, but also chemically modified fats, such as, for example, hydrogenated vegetable oils (for example hydrogenated castor oil and/or hydrogenated coconut fatty glycerides), triglycerides, such as, for example, hydrogenated soy glyceride, trihydroxystearin, fatty acids, fatty acid esters and glycol esters, such as, for example, C₂₀₋₄₀-alkyl stearate, C₂₀₋₄₀-alkyl hydroxystearoylstearate and/or glycol montanate. Also advantageous are furthermore certain organosilicon compounds which have similar physical properties to the specified fat and/or wax components, such as, for example, stearoxytrimethylsilane.

According to the invention, the fat and/or wax components can either be used individually or else as a mixture in the compositions.

Any desired mixtures of such oil and wax components are also to be used advantageously within the context of the present invention.

The oil phase is advantageously selected from the group 2-ethylhexyl isostearate, octyldodecanol, isotridecyl isononanoate, butylene glycol dicaprylate/dicaprate, 2-ethylhexyl cocoate, C₁₂₋₁₅-alkyl benzoate, caprylic/capric acid triglyceride, dicaprylyl ether.

Mixtures of octyldodecanol, caprylic/capric acid triglyceride, dicaprylyl ether, dicaprylyl carbonate, coco glycerides or mixtures of C₁₂₋₁₅-alkyl benzoate and 2-ethylhexyl isostearate, mixtures of C₁₂₋₁₅-alkyl benzoate and butylene glycol dicaprylate/dicaprate, and also mixtures of C₁₂₋₁₅-alkyl benzoate, 2-ethylhexyl isostearate and isotridecyl isononanoate are particularly advantageous.

Of the hydrocarbons, paraffin oil, cycloparaffin, squalane, squalene, hydrogenated polyisobutene and polydecene are to be used advantageously within the context of the present invention.

The oil component is also advantageously selected from the group of phospholipids. The phospholipids are phosphoric acid esters of acylated glycerols.

According to the invention, paraffin oil advantageous according to the invention that can be used is Merkur White Oil Pharma 40 from Merkur Vaseline, Shell Ondina® 917, Shell Ondina® 927, Shell Oil 4222, Shell Ondina®933 from Shell & DEA Oil, Pionier® 6301 S, Pionier® 2071 (Hansen & Rosenthal).

Suitable cosmetically compatible oil and fat components are described in Karl-Heinz Schrader, Grundlagen und Rezepturen der Kosmetika [Fundamentals and Formulations of Cosmetics], 2nd edition, Verlag Huthig, Heidelberg, pp. 319-355, to which reference is made here in its entirety.

The content of further oils, fats and waxes is at most 50% by weight, preferably 30% by weight, further preferably at most 20% by weight, based on the total weight of the composition.

Ethoxylated Glycerol Fatty Acid Esters

The compositions according to the invention comprise, if appropriate, ethoxylated oils selected from the group of ethoxylated glycerol fatty acid esters, particularly preferably PEG-10 olive oil glycerides, PEG-11 avocado oil glycerides, PEG-11 cocoa butter glycerides, PEG-13 sunflower oil glycerides, PEG-15 glyceryl isostearate, PEG-9 coconut fatty acid glycerides, PEG-54 hydrogenated castor oil, PEG-7 hydrogenated castor oil, PEG-60 hydrogenated castor oil, jojoba oil ethoxylate (PEG-26 jojoba fatty acids, PEG-26 jojoba alcohol), glycereth-5 cocoate, PEG-9 coconut fatty acid glycerides, PEG-7 glyceryl cocoate, PEG-45 palm kernel oil glycerides, PEG-35 castor oil, olive oil PEG-7 ester, PEG-6 caprylic acid/capric acid glycerides, PEG-10 olive oil glycerides, PEG-13 sunflower oil glycerides, PEG-7 hydrogenated castor oil, hydrogenated palm kernel oil glyceride PEG-6 ester, PEG-20 corn oil glycerides, PEG-18 glyceryl oleate cocoate, PEG-40 hydrogenated castor oil, PEG-40 castor oil, PEG-60 hydrogenated castor oil, PEG-60 corn oil glycerides, PEG-54 hydrogenated castor oil, PEG-45 palm kernel oil glycerides, PEG-80 glyceryl cocoate, PEG-60 almond oil glycerides, PEG-60 evening primrose glycerides, PEG-200 hydrogenated glyceryl palmate, PEG-90 glyceryl isostearate.

Preferred ethoxylated oils are PEG-7 glyceryl cocoate, PEG-9 coconut glyceride, PEG-40 hydrogenated castor oil, PEG-200 hydrogenated glyceryl palmate.

Active Ingredients

It has been found that active ingredients of varying solubility can be homogeneously incorporated into the compositions according to the invention.

According to the invention, the active ingredients (one or more compounds) can advantageously be selected from the group of NO synthase inhibitors, particularly if the compositions according to the invention are to serve for the treatment and prophylaxis of the symptoms of intrinsic and/or extrinsic aging and also for the treatment and prophylaxis of the harmful effects of ultraviolet radiation on the hair. A preferred NO synthase inhibitor is nitroarginine.

Furthermore, the active ingredients are advantageously selected from the group consisting of catechins and bile acid esters of catechins and aqueous or organic extracts from plants or parts of plants which have a content of catechins or bile acid esters of catechins, such as, for example, the leaves of the plant family Theaceae, in particular of the species Camellia sinensis (green tea). Their typical ingredients (for example polyphenols or catechins, caffeine, vitamins, sugars, minerals, amino acids, lipids) are particularly advantageous.

Catechins are a group of compounds which are to be regarded as hydrogenated flavones or anthocyanidins and are derivatives of “catechins” (catechol, 3,3′,4′,5,7-flavanpentol, 2-(3,4-dihydroxyphenyl)chroman-3,5,7-triol). Epicatechin ((2R,3R)-3,3′,4′,5,7-flavanpentol) is also an advantageous active ingredient within the context of the present invention.

Also advantageous are plant extracts with a content of catechins, in particular extracts of green tea, such as, for example, extracts from leaves of the plants of the Camellia spec. species, very particularly of the tea varieties Camellia sinensis, C. assamica, C. taliensis and C. inawadiensis and hybrids of these with, for example, Camellia japonica.

Preferred active ingredients are also polyphenols and catechins from the group (−) catechin, (+) catechin, (−)-catechin gallate, (−)-gallocatechin gallate, (+)-epicatechin, (−)-epicatechin, (−)-epicatechin gallate, (−)-epigallocatechin, (−)-epigallocatechin gallate.

Flavone and its derivatives (often also collectively called “flavones”) are also advantageous active ingredients within the context of the present invention.

Further advantageous active ingredients are sericoside, pyridoxol, vitamin K, biotin and aroma substances.

Furthermore, the active ingredients (one or more compounds) can also very advantageously be selected from the group of hydrophilic active ingredients, in particular from the following group:

α-hydroxy acids, such as lactic acid or salicylic acid and salts thereof, such as, for example, Nalactate, Ca-lactate, TEA-lactate, urea, allantoin, serine, sorbitol, glycerol, milk proteins, panthenol, chitosan.

The list of specified active ingredients and active ingredient combinations which can be used in the compositions according to the invention is not of course intended to be limiting. The active ingredients can be used individually or in any combinations with one another.

The amount of such active ingredients (one or more compounds) in the compositions according to the invention is preferably 0.001 to 30% by weight, particularly preferably 0.05 to 20% by weight, in particular 1 to 10% by weight, based on the total weight of the composition.

The specified and further active ingredients which can be used in the compositions according to the invention are given in DE 103 18 526 A1 on pages 12 to 17, to which reference is made at this point in its entirety.

Pearlescent Waxes

Suitable pearlescent waxes are, for example: alkylene glycol esters, specifically ethylene glycol distearate (for example Euperlan® PK 710 Benz (BASF)); fatty acid alkanolamides, specifically coconut fatty acid diethanolamide; partial glycerides, specifically stearic acid monoglyceride; esters of polybasic, optionally hydroxy-substituted carboxylic acids with fatty alcohols having 6 to 22 carbon atoms, specifically long-chain esters of tartaric acid; fatty substances, such as, for example, fatty alcohols, fatty ketones, fatty aldehydes, fatty ethers and fatty carbonates which have a total of at least 24 carbon atoms, specifically laurone and distearyl ether; fatty acids such as stearic acid, hydroxystearic acid or behenic acid, ring-opening products of olefin epoxides having 12 to 22 carbon atoms with fatty alcohols having 12 to 22 carbon atoms and/or polyols having 2 to 15 carbon atoms and 2 to 10 hydroxyl groups, and mixtures thereof.

The compositions according to the invention can further comprise glitter substances and/or other effect substances (for example color streaks).

Emulsifiers

In one preferred embodiment, the shampoos compositions according to the invention additionally comprise emulsifiers. Suitable emulsifiers are, for example, nonionogenic surfactants from at least one of the following groups:
(1) addition products of from 2 to 30 mol of ethylene oxide and/or 0 to 5 mol of propylene oxide onto linear fatty alcohols having 8 to 22 carbon atoms, onto fatty acids having 12 to 22 carbon atoms and onto alkylphenols having 8 to 15 carbon atoms in the alkyl group;
(2) C_{12/18} fatty acid mono- and diesters of addition products of from 1 to 30 mol of ethylene oxide onto glycerol;
(3) glycerol mono- and diesters and sorbitan mono- and diesters of saturated and unsaturated fatty acids having 6 to 22 carbon atoms and ethylene oxide addition products thereof;
(4) alkyl mono- and oligoglycosides having 8 to 22 carbon atoms in the alkyl radical and ethoxylated analogs thereof;
(5) addition products of from 15 to 60 mol of ethylene oxide onto oils, for example onto castor oil and/or hydrogenated castor oil;
(6) polyol, and in particular polyglycerol, esters, such as, for example, polyglycerol polyricinoleate, polyglycerol poly-12-hydroxystearate or polyglycerol dimerate. Mixtures of compounds from two or more of these classes of substances are likewise suitable;
(7) addition products of from 2 to 15 mol of ethylene oxide onto castor oil and/or hydrogenated castor oil;
(8) partial esters based on linear, branched, unsaturated or saturated C_{6/22}-fatty acids, ricinoleic acid, and 12-hydroxystearic acid and glycerol, polyglycerol, pentaerythritol, dipentaerythritol, sugar alcohols (for example sorbitol), alkyl glucosides (for example methyl glucoside, butyl glucoside, lauryl glucoside), and polyglucosides (for example cellulose);
(9) mono-, di- and trialkyl phosphates, and mono-, di- and/or tri-PEG alkyl phosphates and salts thereof;
(10) wool wax alcohols;
(11) polysiloxane-polyalkyl-polyether copolymers and corresponding derivatives;
(12) mixed esters of pentaerythritol, fatty acids, citric acid, and fatty alcohol according to DEPS 1165574 and/or mixed esters of fatty acids having 6 to 22 carbon atoms, methylglycose and polyols, preferably glycerol or polyglycerol, and
(13) polyalkylene glycols.

The addition products of ethylene oxide and/or of propylene oxide onto fatty alcohols, fatty acids, alkylphenols, glycerol mono- and diesters, and sorbitan mono- and diesters of fatty acids or onto castor oil are known, commercially available products. These are homolog mixtures whose average degree of alkoxylation corresponds to the ratio of the quantitative amounts of ethylene oxide and/or propylene oxide and substrate with which the addition reaction is carried out. C₁₂₋₁₈-fatty acid mono- and diesters of addition products of ethylene oxide onto glycerol are known from DE C 2024051 as refatting agents for cosmetic preparations. C₈₋₁₈-alkyl mono- and oligoglycosides, their preparation and their use are known from the prior art. Their preparation takes place in particular by reacting glucose or oligosaccharides with primary alcohols having 8 to 18 carbon atoms. As regards the glycoside ester, either monoglycosides in which a cyclic sugar radical is bonded glycosidically to the fatty alcohol, or oligomeric glycosides with a degree of oligomerization up to preferably about 8 are suitable. The degree of oligomerization here is a statistical average value which is based on a homolog distribution customary for such technical products.

Perfume Oils

If appropriate, the compositions according to the invention can comprise perfume oils. Perfume oils which may be mentioned are, for example, mixtures of natural and synthetic fragrances. Natural fragrances are extracts from flowers (lily, lavender, rose, jasmine, neroli, Ylang-Ylang), stems and leaves (geranium, patchouli, petit grain), fruits (anis, coriander, caraway, juniper), fruit peels (bergamot, lemon, orange), roots (mace, angelica, celery, cardamom, costus, iris, calamus), woods (pinewood, sandalwood, guaiac wood, cedarwood, rosewood), herbs and grasses (tarragon, lemongrass, sage, thyme), needles and branches (spruce, fir, pine, dwarf-pine), resins and balsams (galbanum, elemi, benzoe, myrrh, olibanum, opoponax). Also suitable are animal raw materials, such as, for example, civet and castoreum. Typical synthetic fragrance compounds are products of the ester, ether, aldehyde, ketone, alcohol and hydrocarbon type. Fragrance compounds of the ester type are, for example, benzyl acetate, phenoxyethyl isobutyrate, 4-tert-butyl cyclohexyl acetate, linalyl acetate, dimethylbenzylcarbinyl acetate, phenylethyl acetate, linalyl benzoate, benzyl formate, ethylmethyl phenylglycinate, allyl cyclohexylpropionate, styrallyl propionate and benzyl salicylate. The ethers include, for example, benzyl ethyl ether, the aldehydes include, for example, the linear alkanols having 8 to 18 carbon atoms, citral, citronellal, citronellyloxyacetaldehyde, cyclamenaldehyde, hydroxycitronellal, lilial and bourgeonat, the ketones include, for example, the ionones, cc-isomethylions and methyl cedryl ketone, the alcohols include anethiol, citronellol, eugenol, isoeugenol, geraniol, linalool, phenylethyl alcohol and terpineol, and the hydrocarbons include primarily the terpenes and balsams. However, preference is given to using mixtures of different fragrances which together produce a pleasing scent note. Essential oils of lower volatility, which are mostly used as aroma components, are also suitable as perfume oils, for example sage oil, chamomile oil, oil of cloves, melissa oil, mint oil, cinnamon leaf oil, linden blossom oil, juniper berry oil, vetiver oil, olibanum oil, galbanum oil, labolanum oil and lavandin oil. Preference is given to using bergamot oil, dihydromyrcenol, lilial, lyral, citronellol, phenylethyl alcohol, α-hexylcinnamaldehyde, geraniol, benzyl acetone, cyclamenaldehyde, linalool, Boisambrene® Forte, ambroxan, indol, hedione, sandelice, lemon oil, mandarin oil, orange oil, allyl amyl glycolate, cyclovertal, lavandin oil, clary sage oil, β-damascone, geranium oil bourbon, cyclohexyl salicylate, Vertofix® Coeur, iso ESuper®, Fixolide® NP, evernyl, iraldein gamma, phenylacetic acid, geranyl acetate, benzyl acetate, rose oxide, romillat, irotyl and floramat alone or in mixtures.

Pigments

The compositions according to the invention also can comprise pigments. The pigments are present in the product mass in undissolved form and may be present in an amount of from 0.01 to 25% by weight, particularly preferably from 5 to 15% by weight. The preferred particle size is 1 to 200 μm, in particular 3 to 150 μm, and particularly preferably 10 to 100 μm. The pigments are colorants which are virtually insoluble in the application medium and may be inorganic or organic. Inorganic-organic mixed pigments are also possible. Preference is given to inorganic pigments. The advantage of the inorganic pigments is their excellent fastness to light, weather and temperature. The inorganic pigments may be of natural origin, for example prepared from chalk, ocker, umbra, green earth, burnt sienna or graphite. The pigments may be white pigments, such as, for example, titanium dioxide or zinc oxide, black pigments, such as, for example, iron oxide black, colored pigments, such as, for example ultramarine or iron oxide red, luster pigments, metal effect pigments, pearlescent pigments, and fluorescent and phosphorescent pigments, where preferably at least one pigment is a colored, nonwhite pigment.

Metal oxides, hydroxides and oxide hydrates, mixed phase pigments sulfur-containing silicates, metal sulfides, complex metal cyanides, metal sulfates, chromates and molybdates, and also the metals themselves (bronze pigments) are suitable. Of particular suitability are titanium dioxide (CI 77891), black iron oxide (CI 77499), yellow iron oxide (CI 77492), red and brown iron oxide (CI 77491), manganese violet (CI 77742), ultramarine (sodium aluminum sulfosilicates, CI 77007, pigment blue 29), chromium oxide hydrate (C177289), iron blue (ferric ferrocyanide, CI7751 0), carmine (cochineal).

Particular preference is given to pearlescent and colored pigments based on mica which are coated with a metal oxide or a metal oxychloride such as titanium dioxide or bismuth oxychloride, and, if appropriate, further color-imparting substances, such as iron oxides, iron blue, ultramarine, carmine etc., and where the color can be determined by varying the layer thickness. Pigments of this type are sold, for example, under the trade names Rona®, Colorona®, Dichrona® and Timiron® (Merck).

Organic pigments are, for example, the natural pigments sepia, gamboge, charcoal, Cassel brown, indigo, chlorophyll and other plant pigments. Synthetic organic pigments are, for example, azo pigments, anthraquinoids, indigoids, dioxazine, quinacridone, phthalocyanine, isoindolinone, perylene and perinone, metal complex, alkali blue and diketopyrrolopyrrole pigments.

Preferably, the composition according to the invention comprises 0.01 to 10% by weight, particularly preferably from 0.05 to 5% by weight, of at least one particulate substance. Suitable substances are, for example, substances which are solid at room temperature (25° C.) and are present in the form of particles. For example, silica, silicates, aluminates, clay earths, mica, salts, in particular inorganic metal salts, metal oxides, for example titanium dioxide, minerals and polymer particles are suitable.

The particles are present in the composition in undissolved form, preferably in stably dispersed form and, following application to the application surface and evaporation of the solvent, can settle out in solid form.

Preferred particulate substances are silica (silica gel, silicon dioxide) and metal salts, in particular inorganic metal salts, particular preference being given to silica. Metal salts are, for example, alkali metal or alkaline earth metal halides, such as sodium chloride or potassium chloride; alkali metal or alkaline earth metal sulfates, such as sodium sulfate or magnesium sulfate.

Stabilizing Agent

The compositions according to the invention also can comprise at least one stabilizing agent.

Preferred is hydroxyalkylcellulose, wherein alkyl is a C₁-C₄-alkyl, particularly hydroxyethylcellulose. Suitable hydroxyalkylcelluloses can be prepared by alkoxylation of a cellulose material by known methods. Thus, a cellulose can be reacted with ethylene oxide and/propylene oxide. The amount of alkylene oxide is preferably about 0.01 to 5 moles, more preferably about 0.02 to 3.5 moles, especially 0.05 to 2.5 per mole of glucose repeat units in the employed cellulose.

Preferably, the hydroxyalkylcellulose has a degree of polymerization (DP) of 10 to 5000, preferably 20 to 3000, in particular 30 to 1000.

Preferably, the hydroxyalkylcellulose has a degree of substitution with respect to hydroxyalkyl groups (DS) of from 0.01 to 3, more preferably 0.02 to 2, especially 0.02 to 1.5.

Preferred commercially available hydroxyalkylcelluloses are the Natrosol™ trademarks and especially preferred Natrosol™ 250 (CAS-Nr. 9004-62-0) of Herkules Incorporated.

Examples of suitable stabilizing agents are also starches, acrylate homopolymers or acrylate copolymers.

Preferred commercially available starches are sold by National starch, under the trademark National 465, Purity W or starch B990.

Preferred commercially available acrylate polymers or copolymers are Tinovis® CD, Ultragel® 300 and Rheocare® TTA, TTN, TTN-2 (BASF).

When the stabilizing agents are employed, they may be used in an amount of from 0.1 to 5 wt %, preferably from 0.3 to 3 wt %, more preferably from 0.5 to 2.5 wt %, based on the total weight of the compositions.

Needless to say, the composition of the invention should be cosmetically or dermatologically acceptable, i.e., it should contain a non-toxic physiologically acceptable medium and should be able to be applied to the hair of human beings. For the purposes of the invention, the expression “cosmetically acceptable” means a composition of pleasant appearance, odor, feel and/or taste.

The pH of the composition of the invention can be for example from 4.5 to 7, preferably from 4.8 to 6.9, more preferably from 5.0 to 6.7.

Supply Form

It is advantageous if the compositions according to the invention are stored in a tube, a pot, a bottle or squeezable bottle and are applied from this. Accordingly, tubes, pots, bottles or squeezable bottles which comprise a composition according to the invention are also in accordance with the invention.

Another aspect of the present invention relates to a process for preparing the styling shampoo compositions of the present invention, which comprises mixing the components of the compositions.

In one preferred embodiment, the present invention relates to styling shampoo compositions which comprise (a) a cationic polymer with charge density of from 2 to 10 meq/gram, preferably from 4 to 8 meq/gram, more preferably from 5 to 7 meq/gram; (b) at least one anionic surfactant selected from sulfate anionic surfactants and amino acid anionic surfactants; and (c) optionally an amphoteric or non-ionic surfactant or combination thereof, wherein the amount of (a) is from 0.3 to 10 wt %, preferably from 0.5 to 9 wt %, more preferably from 1 to 8 wt %, even more preferably from 1.5 to 8 wt %, based on the weight of the styling shampoo compositions.

In another preferred embodiment, the present invention relates to styling shampoo compositions which comprise (a) a cationic polymer with charge density of from 2 to 10 meq/gram, preferably from 4 to 8 meq/gram, more preferably from 5 to 7 meq/gram; and (b) at least one anionic surfactant selected from amino acid anionic surfactants, wherein the amount of (a) is from 0.3 to 10 wt %, preferably from 0.5 to 9 wt %, more preferably from 1 to 8 wt %, even more preferably from 1.5 to 8 wt %, based on the weight of the styling shampoo compositions, and the amount of (b) is from 7 to 30 wt %, preferably from 8 to 25 wt %, more preferably from 9 to 15 wt %, based on the weight of the styling shampoo compositions.

In another preferred embodiment, the present invention relates to styling shampoo compositions which comprise (a) a cationic polymer (preferably Polyquaternium) with charge density of from 2 to 10 meq/gram, preferably from 4 to 8 meq/gram, more preferably from 5 to 7 meq/gram; and (b) at least one anionic surfactant selected from amino acid anionic surfactants selected from acyl glutamate salts, acyl taurate salts, acyl glycinate salts, acyl alaninate salts, acyl sarcosinate salts and acyl aspartate salts, wherein the amount of (a) is from 0.3 to 10 wt %, preferably from 0.5 to 9 wt %, more preferably from 1 to 8 wt %, even more preferably from 1.5 to 8 wt % based on the weight of the styling shampoo compositions, and the amount of (b) is from 7 to 30 wt %, preferably from 8 to 25 wt %, more preferably from 9 to 15 wt %, based on the weight of the styling shampoo compositions.

In another preferred embodiment, the present invention relates to styling shampoo compositions which comprise (a) a cationic polymer with charge density of from 2 to 10 meq/gram, preferably from 4 to 8 meq/gram, more preferably from 5 to 7 meq/gram; (b) at least one anionic surfactant selected from amino acid anionic surfactants; and (c) an amphoteric or non-ionic surfactant or combination thereof, wherein the amount of (a) is from 0.3 to 10 wt %, preferably from 0.5 to 9 wt %, more preferably from 1 to 8 wt %, even more preferably from 1.5 to 8 wt %, based on the weight of the styling shampoo compositions, the amount of (b) is from 1 to 30 wt %, preferably from 2 to 25 wt %, more preferably from 3 to 20 wt %, even more preferably from 5 to 15 wt %, based on the weight of the styling shampoo compositions, and the amount of (c) is from 0.01 to 20 wt %, preferably 2 to 19 wt %, more preferably from 4 to 15 wt %, based on the weight of the styling shampoo compositions.

In another preferred embodiment, the present invention relates to styling shampoo compositions which comprise (a) a cationic polymer (preferably Polyquaternium) with charge density of from 2 to 10 meq/gram, preferably from 4 to 8 meq/gram, more preferably from 5 to 7 meq/gram; (b) at least one anionic surfactant selected from amino acid anionic surfactants selected from acyl glutamate salts, acyl taurate salts, acyl glycinate salts, acyl alaninate salts, acyl sarcosinate salts and acyl aspartate salts; and (c) an amphoteric and a non-ionic surfactant, wherein the amount of (a) is from 0.3 to 10 wt %, preferably from 0.5 to 9 wt %, more preferably from 1 to 8 wt %, even more preferably from 1.5 to 8 wt %, based on the weight of the styling shampoo compositions, the amount of (b) is from 1 to 30 wt %, preferably from 2 to 25 wt %, more preferably from 3 to 20 wt %, even more preferably from 5 to 15 wt %, based on the weight of the styling shampoo compositions, and the amount of (c) is from 0.01 to 20 wt %, preferably 2 to 19 wt %, more preferably from 4 to 15 wt %, based on the weight of the styling shampoo compositions.

In another preferred embodiment, the present invention relates to styling shampoo compositions which comprise (a) a cationic polymer with charge density of from 2 to 10 meq/gram, preferably from 4 to 8 meq/gram, more preferably from 5 to 7 meq/gram; and (b) at least one anionic surfactant selected from sulfate anionic surfactants, wherein the styling shampoo compositions further comprise acrylate copolymers and the amount of (a) is from 0.3 to 2.5 wt %, preferably from 0.5 to 2.2 wt %, more preferably from 1 to 2 wt %, based on the weight of the styling shampoo compositions, the amount of (b) is from 1 to 30 wt %, preferably from 2 to 25 wt %, more preferably from 3 to 20 wt %, even more preferably from 5 to 15 wt %, based on the weight of the styling shampoo compositions, and the amount of acrylate copolymers is from 0.01 to 3 wt %, preferably from 0.1 to 2.5 wt %, more preferably from 0.2 to 2.1 wt %.

In another preferred embodiment, the present invention relates to styling shampoo compositions which comprise (a) a cationic polymer with charge density of from 2 to 10 meq/gram, preferably from 4 to 8 meq/gram, more preferably from 5 to 7 meq/gram; (b) at least one anionic surfactant selected from sulfate anionic surfactants; and (c) an amphoteric or non-ionic surfactant or combination thereof, wherein the styling shampoo compositions further comprise acrylate copolymers and the amount of (a) is from 0.3 to 2.5 wt %, preferably from 0.5 to 2.2 wt %, more preferably from 1 to 2 wt %, based on the weight of the styling shampoo compositions, the amount of (b) is from 1 to 30 wt %, preferably from 2 to 25 wt %, more preferably from 3 to 20 wt %, even more preferably from 5 to 15 wt %, based on the weight of the styling shampoo compositions, the amount of (c) is from 0.01 to 20 wt %, preferably 2 to 19 wt %, more preferably from 4 to 15 wt %, based on the weight of the styling shampoo compositions, and the amount of acrylate copolymers is from 0.01 to 3 wt %, preferably from 0.1 to 2 wt %, more preferably from 0.2 to 1 wt %.

In another preferred embodiment, the present invention relates to styling shampoo compositions which comprise (a) a cationic polymer with charge density of from 2 to 10 meq/gram, preferably from 4 to 8 meq/gram, more preferably from 5 to 7 meq/gram; (b) at least one anionic surfactant selected from sulfate anionic surfactants; and (c) an amphoteric surfactant, wherein the styling shampoo compositions further comprise acrylate copolymers and the amount of (a) is from 0.3 to 2.5 wt %, preferably from 0.5 to 2.2 wt %, more preferably from 1 to 2 wt %, based on the weight of the styling shampoo compositions, the amount of (b) is from 1 to 30 wt %, preferably from 2 to 25 wt %, more preferably from 3 to 20 wt %, even more preferably from 5 to 15 wt %, based on the weight of the styling shampoo compositions, the amount of (c) is from 0.01 to 20 wt %, preferably 2 to 19 wt %, more preferably from 3 to 15 wt %, based on the weight of the styling shampoo compositions, and the amount of acrylate copolymers is from 0.01 to 3 wt %, preferably from 0.1 to 2 wt %, more preferably from 0.2 to 1 wt %.

### Examples

Materials:

The materials shown in Table 2 are used.

TABLE 2

| Name | INCI Name |
|---|---|
| Cationic | |
| polymer | |
| Luviquat ® | Polyquaternium-16 (38-42%) (and) Water (58-62%) |
| Excellence | Polyquaternium-16: cationic charge density: |
| | 6.1 meq/gram Mw: appr. 40,000 |
| Luviquat ® | Polyquaternium-16 (19-21%) (and) Water (79-81%) |
| HM 552 | Polyquaternium-16: cationic charge density: 3 |
| | meq/gram Mw: appr. 400,000 |
| Hair fixative | |
| Lu-AT3 | VP/Methacrylamide/Vinyl Imidazole Copolymer |
| | (19-21%) (and) Water (79-81%) (and) Preservative: |
| | mixture of Phenoxyethanol (and) Phenylpropanol |
| | (and) Propandiol (and) Caprylyl Glycol (and) |
| | Tocopherol (max. 1%) |
| Amphoteric | |
| surfactant | |
| or non- | |
| ionic surfactant | |
| De-PK 45 | Cocoamidopropyl Betaine (44-46%) (and) Water |
| | (54-56%) |
| PI-818 UP | Coco-Glucoside (50-60%) (and) Water (40-50%) |
| Anionic | |
| surfactant | |
| PI-LGC Sorb | Sodium Lauryl Glucose Carboxylate (15-25%) |
| | (and) Lauryl Glucoside (10-20%) (and) Water |
| | (60-70%) (and) Sodium Citrate (approx. 3.0%) |
| | (and) Sorbic acid (and) (approx. 0.5%) |
| Te-NSO UP | Sodium Laureth Sulfate (20-40%) (and) Water |
| | (60-80%) |
| PI-ACG-LC | Sodium Cocoyl Glutamate (25-35%) (and) Water |
| | (55-65%) (and) Isopropyl Alcohol (max. 2.7%) |
| 30% NANSA ® | Sodium C14-18 Olefin Sulfonate (30%) (and) |
| LSS 495/H | Water (70%) |
| solution | |
| NE-CDT- | Sodium Taurine Cocoyl Methyl Taurate (30%) |
| 30(CR) | (and) Water (70%) |
| Thickener | |
| Rheocare ® | Acrylate Copolymers (29-31%) (and) Water |
| TTA | (69-71%) |
| Comperlan ® | Cocamide MEA (100%) |
| 100 C | |
| Comperlan ® | Cocamide MEA (100%) |
| 100 | |
| Ar-TT | PEG/PPG-120/10 Trimethylolpropane Trioleate |
| | (40-50%) (and) Laueth-2 (40-50%) (and) |
| | Water (approx. 10%) |
| Others | |
| Eu-PK 710 | Glycol Distearate (17-25%) (and) Sodium |
| Benz | Laureth Sulfate (13-17%) (and) Cocamide |
| | MEA (1-5%) (and) Water (55-65%) (and) |
| | Citric Acid (approx. 0.2%) (and) Sodium |
| | Benzoate (approx. 0.5%) |
| 50% Critic | Water (50%) (and) Critic Acid (50%) |
| Acid Solution | |
| GI-Plus | DMDM Hydantoin (and) lodopropynyl |
| | Butylcarbamate |

### Preparation of the Styling Shampoo Compositions

The styling shampoo compositions are prepared as follows and the weight percentages of the materials are shown in Tables 3 to 10.

The cationic polymer is dissolved in water. The non-ionic or amphoteric surfactants are added if needed under stirring. Then the anionic surfactants are added under stirring. To the mixture, other components (thickener, pearlizer, preservative, perfume etc.) are added under stirring. Then pH is adjusted by a pH adjustor (50% critic acid solution) to 5-6.9 (according to Table 3). The mixture is discharged after mixing well.

Foaming Performance Evaluation Method:

Device: Midea Kitchen Blender
1. Prepare 3% shampoo solution with 2.1 g shampoo, 67.9 g tap water.
2. Use blender to stir for 30 s.

All the solution and foam is poured in cylinder and the foam volume is read. The results are shown in Table 3.

Hair Styling Performance Evaluation Method:

Hair Strand Treatment:
1. Weigh the dry hair strand and record the weight as MO.
2. Wet the hair under running tap water. Run the fingers through the hair five times. Pass through each strand once to remove excess water.
3. Weigh 0.67 g basic cleansing shampoo (aqueous solution of Cocamidopropyl Betaine, Sodium Laureth Sulfate, Lauryl Glucoside, and Coco-Glucoside) for each hair strand.
4. Gently massage the shampoo into the hair passing each swatch six times (three times on each side), from the roots to the ends (without creating knots) so as to lather the shampoo.
5. Rinse the swatches under tap water, running the fingers through the hair fifteen times (front 7 times, back 8 times) until all the shampoo is removed.
6. Pass through each strand twice to remove excess water.
7. Comb the strands. If there are knots present in the swatch, comb the swatch from the end to the root, starting with the wide-tooth comb followed by the fine-tooth comb.
8. Repeat operation steps 2-4, except that styling shampoo is used instead of a basic cleansing shampoo.
9. Leave each strand for five minutes.
10. Rinse the strands under tap water, running the fingers through the hair fifteen times (front 7 times, back 8 times) until all the shampoo is removed.
11. Use tissue to absorb the water until 0.8-0.9 g weight uptake vs MO.
12. Use clap to hold the hair strand to make the ends of strands on the top and fix the hair strand by hands 10 times.
13. Take pictures from both slim and wide sides at different time intervals.

Hair Strand Breaking:
1. After more than 24 h and the hair strands are totally dried, take picture to record the status before breaking.
2. Use gummed tape to fix the hair strands onto a mixer (parallel to the table);
3. Put the hair styling breaking frame below the mixer, ensure that the length of hair is about 1 cm longer than the inner side of breaking stick.
4. Set the mixer to 200 rpm, turn the mixer on to break the hair strands by the breaking frame for 30 sec (1 stick).
5. Remove the hair strands and take picture again.

Data Analysis:
1. Use the Image J software to analysis the slim and wide area of hair strands at different time intervals and calculate the surface area. In Image J, first apply “threshold” (with default setting) then apply “analyze particles” (with default setting) to calculate the surface area of hair strands after washing and after breaking.
2. Use the percentage of surface area change between hair strands after washing and after breaking (Δ surface area (after breaking—after washing)/surface area after washing) to evaluate the styling effect of different products. The lower means the better styling effect.

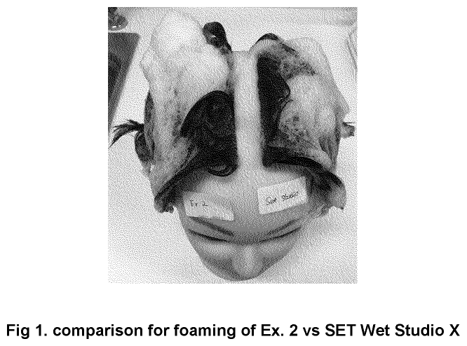

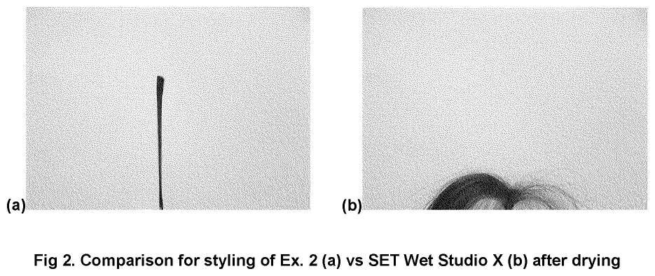

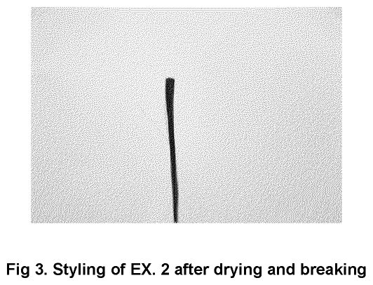

The results are shown in Tables 3 to 9 and FIGS. 1 to 3.

TABLE 3

| Name | | EX. 1 (wt %) | EX. 2 (wt %) |
|---|---|---|---|
| Water | | To 100 | To 100 |
| Luviquat ® Excellence | | | 7.5 |
| Luviquat ® HM 552 | | 30.0 | |
| De-PK 45 | | 6.7 | 9.4 |
| PI-818 UP | | | 14.5 |
| PI-ACG-LC | | | 30.0 |
| Comperlan ® 100 C | | | 2.0 |
| 50% Critic Acid Solution | | q.s. | q.s. |
| GI-Plus | | q.s. | q.s. |
| pH | | 6.7 | 5.0 |
| Appearance | | homogeneous | homogeneous |
| | | & clear | & clear |
| Performance | Foaming (ml) | 250 | 350 |
| data | Styling (%) | 15.86 | 7.17 |

TABLE 4

| | | | COMP. | | COMP. | COMP. |
|---|---|---|---|---|---|---|
| Name | | EX. 3 | EX. 1 | EX. 4 | EX. 2 | EX. 3 |
| Water | | To 100 | To 100 | To 100 | To 100 | To 100 |
| Luviquat® Excellence | | 5.0 | 7.5 | 5.0 | 7.5 | 7.5 |
| Lu-AT3 | | 3.4 | 5.0 | 3.4 | | |
| De-PK 45 | | 17.8 | 17.8 | | | 9.4 |
| PI-818 UP | | | | | | 14.6 |
| 30% NANSA ® | | | | | 33.3 | 36.7 |
| LSS 495/H solution | | | | | | |
| Te-NSO UP | | 37.0 | 37.0 | 37.0 | | |
| Rheocare® TTA | | 3.3 | 3.3 | 7.0 | | |
| Comperlan® 100 C | | 2.0 | 2.0 | 2.0 | | |
| Eu-PK 710 Benz | | 5.0 | 5.0 | 5.0 | | |
| 50% Critic Acid | | q.s. | q.s. | q.s. | q.s. | q.s. |
| Solution | | | | | | |
| GI-Plus | | q.s. | q.s. | q.s. | q.s. | q.s. |
| pH | | 5.7-6 | 5.7-6 | 6.8 | 5.5 | 5.5 |
| Appearance | | homo- | separ- | homo- | separ- | separ- |
| | | geneous | ation | geneous | ation | ation |
| | | & white | | & white | | |
| | | emulsion | | emulsion | | |
| Per- | Foaming | 210 | / | 200 | / | / |
| formance | (ml) | | | | | |
| | Styling | 61.92 | / | 60.11 | / | / |
| data | (%) | | | | | |

TABLE 5

| | | COMP. | | | | | |
|---|---|---|---|---|---|---|---|
| Name | | EX. 4 | EX. 5 | EX. 6 | EX. 7 | EX. 8 | EX. 9 |
| Water | | To 100 | To 100 | To 100 | To 100 | To 100 | To 100 |
| Luviquat ® Excellence | | 7.5 | 7.5 | 7.5 | 12.5 | 5.0 | 20.0 |
| De-PK 45 | | | | 9.4 | 12.2 | 12.2 | 3.6 |
| PI-818 UP | | | | 14.5 | 9.1 | 9.1 | 33.3 |
| PI-ACG-LC | | 20.0 | 36.7 | 20.0 | 36.7 | 50.0 | 16.7 |
| Comperlan ® 100 C | | | | | 2.0 | 2.0 | 2.0 |
| 50% Critic Acid | | q.s. | q.s. | q.s. | q.s. | q.s. | q.s. |
| Solution | | | | | | | |
| GI-Plus | | q.s. | q.s. | q.s. | q.s. | q.s. | q.s. |
| pH | | 5.1 | 6.3 | 6.7 | 5.0 | 5.0 | 5.5 |
| Appearance | | unstable | homogeneous | homogeneous | homogeneous | homogeneous | homogeneous |
| | | | & clear | & clear | & clear | & clear | & clear |
| Performance | Foaming | / | 240 | 280 | 230 | 360 | 250 |
| data | (ml) | | | | | | |
| | Styling | / | 14.98 | 14.69 | 7.76 | 33.59 | 11.71 |
| | (%) | | | | | | |

TABLE 6

| Name | | EX. 10 | EX. 11 |
|---|---|---|---|
| Water | | To 100 | To 100 |
| Luviquat ® Excellence | | 10.0 | 10.0 |
| De-PK 45 | | 15.2 | 15.2 |
| PI-818 UP | | 9.1 | 9.1 |
| PI-ACG-LC | | 33.3 | 33.3 |
| Comperlan ® 100 C | | | |
| Ar-TT | | | 3.0 |
| 50% Critic Acid Solution | | q.s. | q.s. |
| GI-Plus | | q.s. | q.s. |
| pH | | 5.9 | 5.2 |
| Appearance | | homogeneous | homogeneous |
| | | & clear | & clear |
| Performance | Foaming (ml) | 310 | 210 |
| data | Styling (%) | 9.27 | 9.54 |

TABLE 7

| Name | EX. 12 |
|---|---|
| Water | To 100 |
| Luviquat ® Excellence | 7.5 |
| De-PK 45 | 9.4 |
| PI-818 UP | 14.6 |
| NE-CDT-30(CR) | 36.7 |
| 50% Critic Acid Solution | q.s. |
| GI-Plus | q.s. |
| pH | 6.9 |
| Appearance | homogeneous |
| | & clear |

TABLE 8

| Name | COMP. EX. 5 | EX. 13 |
|---|---|---|
| Water | To 100 | To 100 |
| Luviquat ® Excellence | 10.0 | 10.0 |
| De-PK 45 | 15.2 | 15.2 |
| PI-818 UP | 9.1 | 7.0 |
| PI-LGC Sorb | 32.0 | 7.5 |
| PI-ACG-LC | | 28.3 |
| Comperlan ® 100 C | | 3.0 |
| Comperlan ® 100 | 3.0 | |
| 50% Critic Acid Solution | q.s. | q.s. |
| GI-Plus | q.s. | q.s. |
| pH | 5.0 | 5.0 |
| Appearance | separation | homogeneous & clear |

TABLE 9

| Name | EX. 14 |
|---|---|
| Water | To 100 |
| Luviquat ® Excellence | 7.5 |
| PI-818 UP | 22.3 |
| PI-ACG-LC | 36.7 |
| 50% Critic Acid Solution | q.s. |
| GI-Plus | q.s. |
| pH | 5.5 |
| Appearance | homogeneous & clear |

Benchmark's (IGK 1995 2-in-1 Shampoo & Texturizer, SET WET STUDIO X) Evaluation and Result:

SET Wet Studio X—foaming 250 ml, styling 100%

IGK 1995 2-in-1 Shampoo & Texturizer—foaming 370 ml, styling 100%

These two shampoos are commercially available products in the market which claim to have styling effect and comprise cationic polymer as styling agents. It can be seen from the results shown in Tables 3 to 9 and FIGS. 1 to 3 that the styling shampoo compositions of the present invention improve the styling performance of the compositions with equivalent or better foaming, when compared with Benchmarks, which basically has no styling effect.

1. A styling shampoo composition which comprises (a) a cationic polymer with charge density of from 2 to 10 meq/gram; (b) at least one anionic surfactant selected from sulfate anionic surfactants and amino acid anionic surfactants; and (c) optionally an amphoteric or non-ionic surfactant or combination thereof, wherein an amount of (a) is from 0.3 to 10 wt %, based on the weight of the styling shampoo composition.

2. The styling shampoo composition according to claim 1, wherein the styling shampoo composition comprises (a) a cationic polymer with charge density of from 5 to 7; and (b) at least one anionic surfactant selected from sulfate anionic surfactants, wherein the amount of (a) is from 0.3 to 2.5 wt %, based on the weight of the styling shampoo composition.

3. The styling shampoo composition according to claim 1, wherein the styling shampoo composition comprises (a) a cationic polymer with charge density of from 2 to 10 meq/gram; and (b) at least one anionic surfactant selected from amino acid anionic surfactants, wherein the amount of (b) is from 7 to 30 wt %, based on the weight of the styling shampoo composition.

4. The styling shampoo composition according to claim 1, wherein the styling shampoo composition comprises (a) a cationic polymer with charge density of from 2 to 10 meq/gram; (b) at least one anionic surfactant selected from amino acid anionic surfactants; and (c) an amphoteric or non-ionic surfactant or combination thereof, wherein the amount of (b) is from 1 to 30 wt %, based on the weight of the styling shampoo compositions.

5. The styling shampoo composition according to claim 1, wherein the cationic polymer is selected from a copolymer of vinyl monomers having cationic amino or quaternary ammonium functionalities with water soluble spacer monomers.

6. The styling shampoo composition according to claim 1, wherein a weight average molecular weight (Mw) of the cationic polymer is from 10,000 to 5,000,000.

7. The styling shampoo composition according to claim 1, wherein the sulfate anionic surfactant is selected from the group consisting of ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, and combinations thereof.

8. The styling shampoo composition according to claim 1, wherein the amino acid anionic surfactant is selected from the group consisting of acyl glutamate salts, acyl taurate salts, acyl glycinate salts, acyl alaninate salts, acyl sarcosinate salts, and acyl aspartate salts.

9. The styling shampoo composition according to claim 8, wherein the acyl glutamate salts are selected from the group consisting of cocoyl glutamate, lauroyl glutamate, myristoyl glutamate, palmitoyl glutamate, stearoyl glutamate, hydrogenated tallow acyl glutamate, olive oil acyl glutamate and octanoyl glutamate.

10. The styling shampoo composition according to claim 8, wherein the acyl taurate salts are selected from the group consisting of cocoyl taurates, cocoyl methyl taurates, lauric taurates, lauroyl methyl taurates, stearoyl methyl taurates, myristoyl methyl taurates, palmitoyl methyl taurates, oleoyl methyl taurates, hexanoyl methyl taurates, and lauroyl methyl beta-alanine taurates.

11. The styling shampoo composition according to claim 8, wherein the acyl glycinate salts are selected from the group consisting of cocoyl glycinate, palmitoyl glycinate, octanoyl glycinate, and undecylenoyl glycinate.

12. The styling shampoo composition according to claim 8, wherein the acyl alaninate salts are selected from the group consisting of cocoyl alaninate, cocoyl methyl alaninate, lauroyl methyl alaninate, and myristoyl methyl alaninate.

13. The styling shampoo composition according to claim 8, wherein the acyl sarcosinate salts are selected from the group consisting of cocoyl sarcosinate, lauroyl sarcosinate, myristoyl sarcosinate, palmitoyl sarcosinate, and oleoyl sarcosinate.

14. The styling shampoo composition according to claim 8, wherein the acyl aspartate salts are selected from the group consisting of palmitoyl aspartate, myristyl aspartate, lauryl aspartate, and lauroyl aspartate.

15. The styling shampoo composition according to claim 1, wherein the composition further comprises an alkoxylated carboxylate in an amount of from 0.1 to 3% by weight, based on the total weight of the composition.

16. The styling shampoo composition according to claim 15, wherein the alkoxylated carboxylates are selected from the group consisting of Trideceth-7 Carboxylic Acid, Sodium Laureth-13 Carboxylates, Sodium Laureth-4 Carboxylates, Laureth-11 Carboxylic Acid, Laureth-5 Carboxylic Acid, Sodium Laureth-5 Carboxylates, Ammonium Laureth-6 Carboxylates, Ammonium Laureth-8 Carboxylates, Capryleth-4 Carboxylic Acid, Capryleth-6 Carboxylic Acid, Capryleth-9 Carboxylic Acid, Ceteareth-25 Carboxylic Acid, Cetyl C12-15 Pareth-8 Carboxylates, Cetyl C12-15 Pareth-9 Carboxylates, Cetyl PPG-2 Isodeceth-7 Carboxylates, Coceth-7 Carboxylic Acid, C9-11 Pareth-6 Carboxylic Acid, C9-11 Pareth-8 Carboxylic Acid, C11-15 Pareth-7 Carboxylic Acid, C12-13 Pareth-5 Carboxylic Acid, C12-13 Pareth-7 Carboxylic Acid, C12-13 Pareth-8 Carboxylic Acid, C12-13 Pareth-12 Carboxylic Acid, C12-15 Pareth-7 Carboxylic Acid, C12-15 Pareth-8 Carboxylic Acid, C12-15 Pareth-12 Carboxylic Acid, C14-15 Pareth-8 Carboxylic Acid, Deceth-7 Carboxylic Acid, Ethylhexeth-3 Carboxylic Acid, Hexeth-4 Carboxylic Acid, Isopropyl C12-15-Pareth-9 Carboxylate, Isopropyl PPG-2 Isodeceth-7 Carboxylates, Isosteareth-6 Carboxylic Acid, Isosteareth-11 Carboxylic Acid, Laureth-3 Carboxylic Acid, Laureth-4 Carboxylic Acid, Laureth-5 Carboxylic Acid, Laureth-6 Carboxylic Acid, Laureth-8 Carboxylic Acid, Laureth-10 Carboxylic Acid, Laureth-11 Carboxylic Acid, Laureth-12 Carboxylic Acid, Laureth-13 Carboxylic Acid, Laureth-14 Carboxylic Acid, Laureth-17 Carboxylic Acid, Magnesium Laureth-11 Carboxylates, MEA-Laureth-6 Carboxylates, MEA PPG-6 Laureth-7 Carboxylates, MEA-PPG-8-Steareth-7 Carboxylates, Myreth-3 Carboxylic Acid, Myreth-5 Carboxylic Acid, Oleth-3 Carboxylic Acid, Oleth-6 Carboxylic Acid, Oleth-10 Carboxylic Acid, PEG-2 Stearamides Carboxylic Acid, PEG-9 Stearamide Carboxylic Acid, Potassium Laureth-3 Carboxylate, Potassium Laureth-4 Carboxylate, Potassium Laureth-5 Carboxylate, Potassium Laureth-6 Carboxylate, Potassium Laureth-10 Carboxylate, Potassium Trideceth-3 Carboxylate, Potassium Trideceth-4 Carboxylate, Potassium Trideceth-7 Carboxylate, Potassium Trideceth-15 Carboxylate, Potassium Trideceth-19 Carboxylate, PPG-3-Deceth-2 Carboxylic Acid, Propyl C12-15 Pareth-8 Carboxylate, Sodium Capryleth-2 Carboxylate, Sodium Capryleth-9 Carboxylate, Sodium Ceteareth-13 Carboxylate, Sodium Ceteth-13 Carboxylate, Sodium C9-11 Pareth-6 Carboxylates, Sodium C11-15 Pareth-7 Carboxylates, Sodium C12-13 Pareth-5 Carboxylates, Sodium C12-13 Pareth-8 Carboxylates, Sodium C12-13 Pareth-12 Carboxylates, Sodium C12-15 Pareth-6 Carboxylates, Sodium C12-15 Pareth-7 Carboxylates, Sodium C12-15 Pareth-8 Carboxylates, Sodium C12-15 Pareth-12 Carboxylates, Sodium C14-15 Pareth-8 Carboxylates, Sodium C12-14 Sec-Pareth-8 Carboxylates, Sodium Deceth-2 Carboxylates, Sodium Hexeth-4 Carboxylates, Sodium Isosteareth-6 Carboxylates, Sodium Isosteareth-11 Carboxylates, Sodium Laureth-3 Carboxylates, Sodium Laureth-4 Carboxylates, Sodium Laureth-5 Carboxylates, Sodium Laureth-6 Carboxylates, Sodium Laureth-8 Carboxylates, Sodium Laureth-11 Carboxylates, Sodium Laureth-12 Carboxylates, Sodium Laureth-13 Carboxylates, Sodium Laureth-14 Carboxylates, Sodium Laureth-16 Carboxylates, Sodium Laureth-17 Carboxylates, Sodium Lauryl Glucose Carboxylates, Sodium Lauryl Glycol Carboxylate, Sodium PEG-6 Cocamide Carboxylate, Sodium PEG-8 Cocamide Carboxylate, Sodium PEG-3 Lauramide Carboxylate, Sodium PEG-4 Lauramide Carboxylate, Sodium PEG-7 Olive Oil Carboxylate, Sodium PEG-8 Palm Glycerides Carboxylate, Sodium Trideceth-3 Carboxylates, Sodium Trideceth-4 Carboxylates, Sodium Trideceth-6 Carboxylates, Sodium Trideceth-7 Carboxylates, Sodium Trideceth-8 Carboxylates, Sodium Trideceth-12 Carboxylates, Sodium Trideceth-15 Carboxylates, Sodium Trideceth-19 Carboxylates, Sodium Undeceth-5 Carboxylates, Trideceth-3 Carboxylic Acid, Trideceth-4 Carboxylic Acid, Trideceth-7 Carboxylic Acid, Trideceth-8 Carboxylic Acid, Trideceth-15 Carboxylic Acid, Trideceth-19 Carboxylic Acid and undeceth-5 Carboxylic acid, and a combination thereof.

17. The styling shampoo composition according to claim 1, wherein the amphoteric surfactants are selected from the group consisting of sodium acyl amphoacetate, disodium acyl amphodipropionate, disodium alkyl amphodiacetate, sodium acyl amphohydroxypropylsulfonate, disodium acyl amphodiacetate, sodium acyl amphopropionate, and N-coconut fatty acid amidoethyl N-hydroxyethylglycinate sodium salts; N-alkylamino acids, for example aminopropylalkylglutamide, alkylaminopropionic acid, sodium alkylimidodipropionate and lauroamphocarboxyglycinate; alkylbetaines, alkylamidopropylbetaines, alkylsulfobetaines, alkyl glycinates, alkylcarboxyglycinates, alkyl amphoacetates or propionates, and alkyl amphodiacetates or -dipropionates.

18. The styling shampoo composition according to claim 1, wherein the nonionic surfactants are selected from the group consisting of esters which are formed by esterification of carboxylic acids with ethylene oxide, glycerol, sorbitan, and other alcohols, ethoxylated alcohols, ethoxylated lanolin, ethoxylated polysiloxanes, propoxylated POE ethers, alkyl polyglycosides, caprylyl/capryl glucoside, lauryl glucoside, decyl glycoside, coco-glycoside, and glycosides with an HLB value of at least 20.

19. The styling shampoo composition according to claim 1, wherein an amount of the amphoteric or non-ionic surfactants is from 0 to 20 wt %, based on the weight of the styling shampoo composition.

20. The styling shampoo composition according to claim 1, wherein the composition further comprises a thickener.

21. The styling shampoo composition according to claim 20, wherein the thickener is selected from the group consisting of crosslinked polyacrylic acids and derivatives thereof, polysaccharides, guar guar, agar agar, alginates, tyloses, cellulose derivatives, polyethylene glycol mono- and diesters of fatty acids, fatty alcohols, monoglycerides and fatty acids, polyvinyl alcohol, polyvinylpyrrolidone, and alkanolamides.

22. The styling shampoo composition according to claim 1, wherein the further comprises a stabilizing agent.

23. The styling shampoo according to claim 22, wherein the stabilizing agent is selected from the group consisting of hydroxyalkylcellulose, wherein alkyl is a C₁-C₄-alkyl, starches, acrylate homopolymers and acrylate copolymers.

24. The styling shampoo composition according to claim 1, wherein a pH of the composition is from 4.5 to 7.

25. A process for preparing a styling shampoo composition as defined in claim 1, which comprises mixing the components of the in a following order: (a) dissolving the cationic polymer in water; (b) adding the optional non-ionic or amphoteric surfactants; (c) adding the anionic surfactants; (d) adding other components.

26. (canceled)

27. A method of cleaning and styling human hair comprising applying a styling shampoo composition as defined in claim 1 to the human hair.
